(19) United States
(12) Patent Application Publication
(10) Pub. No.: US 2008/0038236 A1
(43) Pub. Date: Feb. 14, 2008

(54) BIOCOMPATIBLE SCAFFOLDS AND ADIPOSE-DERIVED STEM CELLS

(75) Inventors: Jeffrey M. Gimble, Chapel Hill, NC (US); David L. Kaplan, (US); Joshua R. Mauney, (US)

Correspondence Address: SEED INTELLECTUAL PROPERTY LAW GROUP PLLC, 701 FIFTH AVE, SUITE 5400, SEATTLE, WA, 98104, US

(73) Assignee: Artecel Sciences, inc., Durham, NC (US)

(21) Appl. No.: 11/682,767

(22) Filed: Mar. 6, 2007

Related U.S. Application Data
(60) Provisional application No. 60/779,616, filed on Mar. 6, 2006.

(51) Int. Cl. A61K 45/00; A61P 19/08

(52) U.S. Cl. 424/93.21; 424/93.7

(57) ABSTRACT

The present invention relates to compositions of biocompatible materials and adult stem cells. The present invention also provides methods of alleviating or treating bone defects or soft tissue defects using the compositions.

### CROSS-REFERENCE TO RELATED APPLICATION

This application claims the benefit under 35 U.S.C. §119(e) of U.S. Provisional Patent Application No. 60/779,616 filed Mar. 6, 2006, where this provisional application is incorporated herein by reference in its entirety.

### STATEMENT OF GOVERNMENT INTEREST

This invention was made with government support under Grant No. EB002520 awarded by the National Institutes of Health. The government has certain rights in this invention.

### BACKGROUND OF THE INVENTION

1. Field of the Invention

The present invention is directed to the field of stem cells and their use in the treatment of disease and injury, including biocompatible matrices comprising stem cells and their use.

2. Description of the Related Art

Tissue engineering and regenerative medicine seek to combine biomaterials, growth factors and cells to create novel therapeutics to repair damaged tissue and organs. The use of multipotential stem cells is typically envisioned for tissue engineering. Such tissue-engineered therapeutics are expected to have multiple applications, including orthopedic, plastic and reconstructive applications. See, for instance, Patrick, 2001, Anat. Rec. 263:361-366. The use of a patient's own multipotential stem cells (autologous) in tissue engineering applications has numerous hypothetical advantages, most notably the lack of immune reaction to the tissue-engineered therapeutic or by the therapeutic against the host, which can cause graft rejection as well as a variety of difficult, and sometimes life-threatening, side effects. However, the autologous approach requires sufficient time to harvest and expand the patient's stem cells. If the patient's condition is not stable, this approach is likely to be ineffectual as a treatment.

The current paradigms of adult stem cell transplantation are anchored by pioneering investigations involving hematopoietic stem cells, which express both Class I and Class II Major Histocompatibility Antigens. It is well established that the immune system uses the combined presence of MHC Class I and Class II to distinguish between self from non-self. A mismatch in the MHC Class II of donor and host increases the likelihood of rejection. Unlike hematopoietic stem cells, neither adipose-derived stem cells (ASCs) or bone-marrow stromal cells (BMSCs), both of which are adult stem cells, express the MHC Class II molecule in their undifferentiated state. A growing body of literature, based on in vitro and in vivo studies, supports the hypothesis that ASCs and BMSCs can be transplanted across classical histocompatibility barriers with reduced risk of immune response. If correct, this observation will accelerate the pace of discovery in tissue engineering and the manufacture, quality control, and distribution of allogeneic (non-self) adult stem cells for immediate use at the point of care, e.g. emergency care, would become possible.

Tissue engineering typically involves the use of matrices to support the proliferation of cells seeded on it. A number of biocompatible materials have been used or suggested for such matrices, including poly L lactic acid (PLA), polyglycolic acid (PGA), poly DL lactic-co-glycolic acid (PLGA), polycarbonate, hyluronate and collagen-based materials. Silk, a natural polymer, has recently been suggested for use in matrices for tissue engineering (Meinel et al., 2005, Biomaterials 26(2):147-55).

Silk fibers are polymers of the protein fibroin made by the silkworm, Bombyx mori, as well as a large number of spiders. Silk possesses many properties that are particularly useful for tissue engineering and regeneration. Silks have been used as FDA-approved sutures for decades, are biocompatible and are less immunogenic and inflammatory than collagens or polyesters such as PLGA (Altman et al., 2003, Biomaterials 24:401-416; Panilaitis et al., 2003, Biomaterials 24:3079-3085 and Meinel et al., 2004, Biotechnol. Bioeng. 88:379-391). Silk provides a robust mechanical integrity. Native silk fibers exhibit strength, flexibility and resistance to mechanical compression that exceed all other natural fibers and also rival even synthetic high performance fibers. See, for instance, Mahoney et al., 1994, In “Silk Polymers: Materials Science and Biotechnology”, Kaplan et al., Eds, Am. Chem. Soc. Symp. Series, Washington, DC Vol. 544, pp. 196-210. These features are particularly important for in situ repairs where there is a need to form a matrix and retain mechanical integrity during, for instance, osseo-reintegration. Thermal stability is also a hallmark of silk-based biomaterials; they can be autoclaved without loss of mechanical integrity (see, for instance, Altman et al., 2003, Biomaterials 24:401-416). Furthermore, the biodegradation rate of silk is slow enough to maintain porosity and transport for cell ingrowth, survival and new tissue formation, for mechanical integrity, and as templates for in situ mineralization. Silk may be processed into a variety of different biomaterial formats, can be functionalized and is capable of self-assembly permitting conformal fill-ins in vivo during tissue regeneration.

Despite the work summarized above, little progress has been made in defining matrices appropriate for use with adipose-derived stem cells or bone-marrow-derived mesenchymal stem cells. Consequently, there is a clear need in the art for tissue engineered materials that are fully biocompatible, provide appropriate mechanical features for the designated applications and permit the use of allogeneic cells. The present invention addresses and meets these needs.

### BRIEF DESCRIPTION OF THE DRAWINGS

For the purpose of illustrating the invention, there are depicted in the drawings certain embodiments of the invention. However, the invention is not limited to the precise arrangements and instrumentalities of the embodiments depicted in the drawings.

FIG. 1 is a series of images and a graph relating to induction of adipogenesis in ASCs. FIG. 1A is an image of ASCs that were not cultured in adipogenic medium. FIG. 1B is an image of ASCs cultured in adipogenic medium. Confluent stromal cell cultures were induced for 3 days with dexamethasone, insulin, isobutylmethylxanthine and a thiazolidinedione followed by culture in the presence of dexamethasone and insulin. After a total of 14 days in culture, the cells were fixed and stained for neutral lipid with Oil Red O. FIG. 1C is a graph of leptin levels in the conditioned medium from the control cells (Pre-AD) and adipogenic cultures (AD). Leptin levels were determined by ELISA on successive days.

FIG. 2 is a graph of the in vitro immunogenicity of various populations of cells as measured by mixed lymphocyte reactions (MLRs). CPM measures tritiated thymidine incorporation into periperal blood mononuclear cells (PBMCs). Auto PBMCs are autologous peripheral blood mononuclear cells. Allo PBMCs are allogeneic peripheral blood mononuclear cells. SVF are stromal vascular fraction ASCs. P0 indicates ASCs that have not been passaged (i.e. primary culture). P1-P4 are ASCs that have been passaged once, twice, thrice and four-times, respectively. * indicates significant difference in response compared to the response induced by Auto PBMCs, using Student's t test (ΔCPM>750; Stimulation Index>3; p<0.05).

FIGS. 3A-3H are a series of images and graphs of the characterization of human BMSCs (hBMSCs) subjected to chondrogenesis or osteogenesis. FIG. 3A is an image of a phase-contrast photomicrograph of passage 2 hBMSCs (20× magnification). FIGS. 3B and 3C are images of hBMSCs pellets cultured in chondrogenic or control medium, respectively, and stained with safranin O/Fast Red. Pellet diameter ˜2 mm. FIG. 3D is a bar graph of sulphated GAG/DNA (μg/μg) deposition of passages 1, 3, and 5 hBMSCs after 4 weeks of culture in chondrogenic medium. Data is average±standard deviation, n=5 pellets. FIG. 3E is a graph of endoglin (CD105) expression of passage 2 hBMSCs. FIGS. 3F and 3G are images of hBMSC pellets cultured in either osteogenic or control medium, respectively, and stained according to von Kossa. Pellet diameter ˜2 mm. FIG. 3H is a graph of calcium deposition/DNA (μg/ng) of passages 1, 3 and 5 hBMSCs pellet culture in osteogenic medium. Passage 1 and 3 hBMSCs deposited significantly more calcium/DNA than passage 5 cells (p<0.05). Data is average±standard deviation, n=5 pellets.

FIGS. 4A and 4B are graphs of biochemical characterization data from hBMSC differentiation on collagen, silk, and silk-RDG scaffolds after 2 and 4 weeks of culturing in osterogenic medium. FIG. 4A is calcium deposition per scaffold data. FIG. 4B is alkaline phosphatase (AP) activity per scaffold data. Data are average±standard deviation of 3-4 scaffolds. (p<0.05=*; p<0.01=**).

FIGS. 5A-5L are a series of images of histological sections of hBMSC-seeded scaffolds after 2 weeks (upper two rows) or 4 weeks (lower two rows) of culturing in osteogenic medium. FIGS. 5A-5D are sections of collagen scaffolds. FIGS. 5E-5H are sections of silk scaffolds. FIGS. 5I-5L are sections of silk-RGD scaffolds. Von Kossa staining (5A, 5C, 5E, 5G, 5I, 5K) and H&E staining (5B, 5D, 5F, 5H, 5J, 5L). bar=70 μm. Arrows indicate calcification; asterisks indicate polymer; O=osteoblast-like cell, F=fibroblast-like cell, B=collagen-like bundles.

FIGS. 6A-6D are a series of MicroCT images from collagen (FIGS. 6A and 6B) and silk-RGD (FIGS. 6C and 6D) scaffolds. FIGS. 6A and 6C are cross views; FIGS. 6B and 6D are face views of scaffolds. Insert in 6C is a magnification from FIG. 6D. Bar length=1.1 mm.

FIGS. 7A-7T are a series of representative histological and immunohistochemical microphotographs for hematoxylin and eosin (7A-7E), bone sialoprotein (7F-7J), osteocalcin (7K-7O) and osteopontin (7P-7T) of calvarial bone critical size defects in mice after different implantation treatments. Tissue-engineered grafts with scaffolds loaded with BMP-2 (7A, 7F, 7K, 7P), grafts with scaffolds loaded with BMP-2 and seeded with hBMSCs (7B, 7G, 7L, 7Q), scaffolds loaded with BMP-2 alone (7C, 7H, 7M, 7R), plain scaffolds (7D, 7I, 7N, 7S) and empty defects (7E, 7J, 7O 7T). Black arrows indicate host bone around the defect where it could be distinguished. In some cases, implants integrated so well that it was difficult to assess surrounding host bone because it could not be identified separate from the integrated implants.

FIGS. 8A-8D are a series of X-ray (top row) and MicroCT (bottom row) images of mouse calvaria 5 weeks after transplant surgery. FIG. 8A is an X-ray image of a mouse calvarium in which the left calvarial defect was filled with tissue-engineered graft comprising a 3-dimensional silk scaffold. The right defect was not filled. FIG. 8B is an X-ray image of a mouse calvarium in which the left defect was filled with a graft comprising a 3-dimensional silk scaffold seeded with hBMSCs; the graft was not tissue-engineered (i.e. not cultured in osteogenic medium). The right defect was not filled. FIG. 8C is a MicroCT image of a full calvarium in which the left defect was filled with tissue-engineered graft. The right defect was not filled. FIG. 8D is a close-up image of a calvarial defect filled with tissue-engineered graft and depicts the bone of the graft and integration around the periphery of the defect.

### DETAILED DESCRIPTION OF THE INVENTION

The present invention springs from the observation that adult stem cells having multipotentiality, having very low to no immunogenicity, may be seeded on biocompatible scaffolds and can be induced to undergo differentiation along specific lineage pathways. The present invention thus provides compositions of adult stem cells and biocompatible scaffolds. The invention further provides methods of making the compositions. The invention further features methods of alleviating or treating tissue defects, including bone defects and soft tissue defects, using the compositions.

### DEFINITIONS

Unless defined otherwise, all technical and scientific terms used herein generally have the same meaning as commonly understood by one of ordinary skill in the art to which this invention belongs. Generally, the nomenclature used herein and the laboratory procedures in cell culture, molecular genetics, organic chemistry, and nucleic acid chemistry and hybridization are those well known and commonly employed in the art.

Standard techniques are used for nucleic acid and peptide synthesis. The techniques and procedures are generally performed according to conventional methods in the art and various general references (e.g., Sambrook and Russell, 2001, Molecular Cloning, A Laboratory Approach, Cold Spring Harbor Laboratory Press, Cold Spring Harbor, N.Y., and Ausubel et al., 2002, Current Protocols in Molecular Biology, John Wiley & Sons, New York, N.Y.), which are provided throughout this document.

The articles “a” and “an” are used herein to refer to one or to more than one (i.e., to at least one) of the grammatical object of the article. By way of example, “an element” means one element or more than one element.

The term “about” will be understood by persons of ordinary skill in the art and will vary to some extent based on the context in which it is used.

As used herein, “in vitro” and “ex vivo” are used interchangeably to refer to conditions outside the body of a living organism. Thus, in vitro culturing and ex vivo culturing both refer to culturing outside the body of a living organism.

“Adipose” refers to any fat tissue. The adipose tissue may be brown, yellow or white adipose tissue. Preferably, the adipose tissue is subcutaneous white adipose tissue. Adipose tissue includes adipocytes and stroma. Adipose tissue is found throughout the body of an animal. For example, in mammals, adipose tissue is present in the omentum, bone marrow, subcutaneous space, fat pads (e.g., scapular or infrapatellar fat pads), and surrounding most organs. Cells obtained from adipose tissue may comprise a primary cell culture or an immortalized cell line. The adipose tissue may be from any organism having fat tissue. Preferably the adipose tissue is from a primate, more preferably from a mammal, and most preferably the adipose tissue is from a human. A convenient and abundant source of human adipose tissue is that derived from liposuction surgery. However, the source of adipose tissue or the method of isolation of adipose tissue are not critical to the invention.

The term “adipose tissue-derived cell” refers to a cell that originates from adipose tissue. The initial cell population isolated from adipose tissue is a heterogenous cell population including, but not limited to stromal vascular fraction (SVF) cells.

As used herein, the term “adipose derived stromal cells,” “adipose tissue-derived stromal cells,” “adipose tissue-derived adult stromal (ADAS) cells,” or “adipose-derived stem cells” (ASCs) are used interchangeably and refer to stromal cells that originate from adipose tissue which can serve as stem cell-like precursors to a variety of different cell types such as, but not limited to, adipocytes, osteocytes, chondrocytes, muscle and neuronal/glial cell lineages. ASCs are a subset population derived from adipose tissue which can be separated from other components of the adipose tissue using standard culturing procedures or other methods disclosed herein. In addition, ASCs may be isolated from a mixture of cells based on the cell surface markers disclosed herein.

The terms “precursor cell,” “progenitor cell,” and “stem cell” are used interchangeably in the art and as used herein refer either to a pluripotent or lineage-uncommitted progenitor cell, which is potentially capable of an unlimited number of mitotic divisions to either renew itself or to produce progeny cells which will differentiate into the desired cell type. In contrast to pluripotent stem cells, lineage-committed progenitor cells are generally considered to be incapable of giving rise to numerous cell types that phenotypically differ from each other. Instead, progenitor cells give rise to one or possibly two lineage-committed cell types.

As used herein, the term “multipotential” or “multipotentiality” is meant to refer to the capability of a stem cell to differentiate into more than one type of cell.

As used herein, the term “late passaged adipose tissue-derived stromal cell,” refers to a cell exhibiting a less immunogenic characteristic when compared to an earlier passaged cell. The immunogenicity of an adipose tissue-derived stromal cell corresponds to the number of passages. Preferably, the cell has been passaged up to at least the second passage, more preferably, the cell has been passaged up to at least the third passage, and most preferably, the cell has been passaged up to at least the fourth passage.

As used herein, “scaffold” refers to a structure, comprising a biocompatible material, that provides a surface suitable for adherence and proliferation of cells. A scaffold may further provide mechanical stability and support. A scaffold may be in a particular shape or form so as to influence or delimit a three-dimensional shape or form assumed by a population of proliferating cells. Such shapes or forms include, but are not limited to, films (e.g. a form with two-dimensions substantially greater than the third dimension), ribbons, cords, sheets, flat discs, cylinders, spheres, 3-dimensional amorphous shapes, etc.

As used here, “biocompatible” refers to any material, which, when implanted in a mammal, does not provoke an adverse response in the mammal. A biocompatible material, when introduced into an individual, is not toxic or injurious to that individual, nor does it induce immunological rejection of the material in the mammal.

As used herein, “autologous” refers to a biological material derived from the same individual into whom the material will later be re-introduced.

As used herein, “allogeneic” refers to a biological material derived from a genetically different individual of the same species as the individual into whom the material will be introduced.

As used herein, a “graft” refers to a cell, tissue or organ that is implanted into an individual, typically to replace, correct or otherwise overcome a defect. A graft may further comprise a scaffold. The tissue or organ may consist of cells that originate from the same individual; this graft is referred to herein by the following interchangeable terms: “autograft”, “autologous transplant”, “autologous implant” and “autologous graft”. A graft comprising cells from a genetically different individual of the same species is referred to herein by the following interchangeable terms: “allograft”, “allogeneic transplant”, “allogeneic implant” and “allogeneic graft”. A graft from an individual to his identical twin is referred to herein as an “isograft”, a “syngeneic transplant”, a “syngeneic implant” or a “syngeneic graft”. A “xenograft”, “xenogeneic transplant” or “xenogeneic implant” refers to a graft from one individual to another of a different species.

As used herein, the terms “tissue grafting” and “tissue reconstructing” both refer to implanting a graft into an individual to treat or alleviate a tissue defect, such as a bone defect or a soft tissue defect.

As used herein, to “alleviate” a disease, defect, disorder or condition means reducing the severity of one or more symptoms of the disease, defect, disorder or condition.

As used herein, to “treat” means reducing the frequency with which symptoms of a disease, defect, disorder, or adverse condition, and the like, are experienced by a patient.

As used herein, a “therapeutically effective amount” is the amount of a composition of the invention sufficient to provide a beneficial effect to the individual to whom the composition is administered.

As used herein, “bone defect” refers to bone that is broken, fractured, missing portions or otherwise damaged. Such damage may be due to congenital anomaly, disease, disease treatment, trauma or osseous infection, and may be acute or chronic. For instance, bone loss may occur as a result of tumor resection, thus resulting in a bone defect. Non-limiting examples of bone defects include: bone fractures, bone/spinal deformation, osteosarcoma, myeloma, bone dysplasia, scoliosis, osteroporosis, osteomalacia, rickets, fibrous osteitis, fibrous dysplasia, renal bone dystrophy, and Paget's disease of bone.

As used herein, “soft tissue defect” refers to soft tissue that is missing, reduced in quantity or otherwise damaged. A soft tissue defect may result from a congenital anomaly, disease, disease treatment, or trauma, and may be acute or chronic. For instance, a masectomy results in a soft tissue defect. As used herein, soft tissue defect also includes defects that are partially or solely cosmetic. For instance, breast augmentation, lip augmentation and wrinkle removal via injection of a soft tissue filler are all considered treatments for a soft tissue defect.

As used herein, the term “growth medium” is meant to refer to a culture medium that promotes growth of cells. A growth medium will generally contain animal serum. In some instances, the growth medium may not contain animal serum.

“Differentiation medium” is used herein to refer to a cell growth medium comprising an additive or a lack of an additive such that a stem cell, adipose derived adult stem cell or other such progenitor cell, that is not fully differentiated when incubated in the medium, develops into a cell with some or all of the characteristics of a differentiated cell.

By “growth factors” is intended the following specific factors including, but not limited to, growth hormone, erythropoietin, thrombopoietin, interleukin 3 (IL-3), interleukin 6 (IL-6), interleukin 7 (IL-7), macrophage colony stimulating factor, c-kit ligand/stem cell factor, osteoprotegerin ligand, insulin, insulin like growth factors, epidermal growth factor (EGF), fibroblast growth factor (FGF), nerve growth factor, ciliary neurotrophic factor, platelet derived growth factor (PDGF), and bone morphogenetic protein (BMP) at concentrations of between picogram/ml to milligram/ml levels.

“Immunophenotype” of a cell is used herein to refer to the phenotype of a cell in terms of the surface protein profile of a cell.

An “isolated cell” refers to a cell which has been separated from other components and/or cells which naturally accompany the isolated cell in a tissue or mammal.

As used herein, a “substantially purified” cell is a cell that is essentially free of other cell types. Thus, a substantially purified cell refers to a cell which has been purified from other cell types with which it is normally associated in its naturally occurring state.

“Expandability” is used herein to refer to the capacity of a cell to proliferate, for example, to expand in number or, in the case of a population of cells, to undergo population doublings.

“Proliferation” is used herein to refer to the reproduction or multiplication of similar forms, especially of cells. That is, proliferation encompasses production of a greater number of cells, and can be measured by, among other things, simply counting the numbers of cells, measuring incorporation of ³H-thymidine into the cell, and the like.

As used herein, the term “non-immunogenic” refers to the property of a cell to not induce proliferation of T cells, either in vitro in an MLR or in vivo.

As used herein, “tissue engineering” refers to the process of generating tissues ex vivo for use in tissue replacement or reconstruction. Tissue engineering is an example of “regenerative medicine”, which encompasses approaches to the repair or replacement of tissues and organs by incorporation of cells, gene or other biological building blocks, along with bioengineered materials and technologies.

As used herein “endogenous” refers to any material from or produced inside an organism, cell or system.

“Exogenous” refers to any material introduced into or produced outside an organism, cell, or system.

“Encoding” refers to the inherent property of specific sequences of nucleotides in a polynucleotide, such as a gene, a cDNA, or an mRNA, to serve as templates for synthesis of other polymers and macromolecules in biological processes having either a defined sequence of nucleotides (i.e., rRNA, tRNA and mRNA) or a defined sequence of amino acids and the biological properties resulting therefrom. Thus, a gene encodes a protein if transcription and translation of mRNA corresponding to that gene produces the protein in a cell or other biological system. Both the coding strand, the nucleotide sequence of which is identical to the mRNA sequence and is usually provided in sequence listings, and the non-coding strand, used as the template for transcription of a gene or cDNA, can be referred to as encoding the protein or other product of that gene or cDNA.

Unless otherwise specified, a “nucleotide sequence encoding an amino acid sequence” includes all nucleotide sequences that are degenerate versions of each other and that encode the same amino acid sequence. Nucleotide sequences that encode proteins and RNA may include introns.

An “isolated nucleic acid” refers to a nucleic acid segment or fragment which has been separated from sequences which flank it in a naturally occurring state, i.e., a DNA fragment which has been removed from the sequences which are normally adjacent to the fragment, i.e., the sequences adjacent to the fragment in a genome in which it naturally occurs. The term also applies to nucleic acids which have been substantially purified from other components which naturally accompany the nucleic acid, i.e., RNA or DNA or proteins, which naturally accompany it in the cell. The term therefore includes, for example, a recombinant DNA which is incorporated into a vector, into an autonomously replicating plasmid or virus, or into the genomic DNA of a prokaryote or eukaryote, or which exists as a separate molecule (i.e., as a cDNA or a genomic or cDNA fragment produced by PCR or restriction enzyme digestion) independent of other sequences. It also includes a recombinant DNA which is part of a hybrid gene encoding additional polypeptide sequence.

In the context of the present invention, the following abbreviations for the commonly occurring nucleic acid bases are used. “A” refers to adenosine, “C” refers to cytosine, “G” refers to guanosine, “T” refers to thymidine, and “U” refers to uridine.

The phrase “under transcriptional control” or “operatively linked” as used herein means that the promoter is in the correct location and orientation in relation to the polynucleotides to control RNA polymerase initiation and expression of the polynucleotides.

As used herein, the term “promoter/regulatory sequence” means a nucleic acid sequence which is required for expression of a gene product operably linked to the promoter/regulatory sequence. In some instances, this sequence may be the core promoter sequence and in other instances, this sequence may also include an enhancer sequence and other regulatory elements which are required for expression of the gene product. The promoter/regulatory sequence may, for example, be one which expresses the gene product in a tissue specific manner.

A “constitutive” promoter is a nucleotide sequence which, when operably linked with a polynucleotide which encodes or specifies a gene product, causes the gene product to be produced in a cell under most or all physiological conditions of the cell.

An “inducible” promoter is a nucleotide sequence which, when operably linked with a polynucleotide which encodes or specifies a gene product, causes the gene product to be produced in a cell substantially only when an inducer which corresponds to the promoter is present in the cell.

A “tissue-specific” promoter is a nucleotide sequence which, when operably linked with a polynucleotide which encodes or specifies a gene product, causes the gene product to be produced in a cell substantially only if the cell is a cell of the tissue type corresponding to the promoter.

A “vector” is a composition of matter which comprises an isolated nucleic acid and which can be used to deliver the isolated nucleic acid to the interior of a cell. Numerous vectors are known in the art including, but not limited to, linear polynucleotides, polynucleotides associated with ionic or amphiphilic compounds, plasmids, and viruses. Thus, the term “vector” includes an autonomously replicating plasmid or a virus. The term should also be construed to include non-plasmid and non-viral compounds which facilitate transfer of nucleic acid into cells, such as, for example, polylysine compounds, liposomes, and the like. Examples of viral vectors include, but are not limited to, adenoviral vectors, adeno-associated virus vectors, retroviral vectors, and the like.

“Expression vector” refers to a vector comprising a recombinant polynucleotide comprising expression control sequences operatively linked to a nucleotide sequence to be expressed. An expression vector comprises sufficient cis-acting elements for expression; other elements for expression can be supplied by the host cell or in an in vitro expression system. Expression vectors include all those known in the art, such as cosmids, plasmids (i.e., naked or contained in liposomes) and viruses that incorporate the recombinant polynucleotide.

### DESCRIPTION OF THE INVENTION

In the present invention, it is demonstrated that biocompatible scaffolds can be seeded with adult stem cells and the resultant compositions can be used for tissue reconstruction in vivo. The adult stem cells may be induced to differentiate prior to implantation for tissue reconstruction (i.e. ex vivo) or may be induced to differentiate after implantation (i.e. in vivo). In one embodiment, silk fibroin can be used to make a porous biocompatible scaffold which is seeded with ASCs. After seeding, the cells on the silk scaffold are, optionally, subjected to an expansion medium or to a differentiation medium (e.g. osteogenic or adipogenic medium) in vitro. The composition is then implanted into an animal subject in need of tissue reconstruction. The implanted composition supports additional cell growth in vivo, thus providing tissue reconstruction. Advantageously, ASCs have been shown to have very low immunogencity. That is, a composition including ASCs, when transplanted into an allogeneic subject, does not induce, or induces a very minimal immune response. Consequently, the subject does not require immunosuppressant drugs or the requirement is significantly reduced.

The subject may be a mammal, but is preferably a human and the source of the cells for growth and implantation is any mammal, preferably a human.

The present application, therefore, features a composition comprising a silk scaffold and an adult stem cell. The present invention further features methods of making the composition, and methods of using the composition for tissue reconstruction or tissue grafting therapies.

The compositions and methods of the instant invention have myriad useful applications. The compositions may be used in therapeutic methods for alleviating or treating tissue defects in an individual. The compositions may also be used in vitro or in vivo to identify compounds that induce or inhibit specific differentiation pathways, or affect repair of tissue defects, and therefore may have therapeutic potential.

I. Isolating and Expanding Adipose-Derived Stem Cells

The compositions and methods of the instant invention can be practiced using an adipose-derived stem cell from any animal. Preferably, the animal is a mammal, more preferably a primate and more preferably still, a human.

The ASCs useful in the methods of the present invention may be isolated by a variety of methods known to those skilled in the art. ASCs are isolated from a sample of adipose tissue. For example, methods of isolating ASCs are described in U.S. Pat. No. 6,153,432; Aust et al., 2004, Cytotherapy 6:7-14; Halvorsen et al., 2001, Metabolism 50: 407-413; Sen et al., 2001, J Cell Biochem. 81: 312-319; and Gimble et al., 2003, Cytotherapy 5:362-369, each of which are incorporated herein in its entirety.

The immunophenotype of ASCs changes progressively, depending on culturing procedures (i.e. passage number). The adherence to plastic and subsequent expansion of human adipose-derived cells selects for a relatively homogeneous cell population, enriching for cells expressing a “stromal” immunophenotype, as compared to the heterogeneity of the crude stromal vascular fraction. ASCs also express stem-cell associated markers including, but not limited to, human multidrug transporter (ABCG2) and aidehyde dehydrogenase (ALDH).

The immunophenotype of ASCs can be exploited to serve as unique identifiers for ASCs. That is, the unique cell surface markers on the cells of interest can be used to isolate a specific sub-population of cells from a mixed population of cells derived from adipose tissue. One skilled in the art would appreciate that an antibody specific for a cell surface marker can be conjugated to a physical support (i.e. a streptavidin bead) and therefore be used to bind and isolate ASCs having that specific cell surface marker. An example of an antibody that specifically binds to an ASC includes, but is not limited to, anti-ABCG2 antibody. After binding, the bound ASCs can be separated from the remaining cells by, for instance, magnetic separation using magnetic beads, including but not limited to Dynabeads® (Dynal Biotech, Brown Deer, Wis.). Further to the use of Dynabeads®, MACS separation reagents (Miltenyi Biotec, Auburn, Calif.) can be used to remove ASCs from a mixed population of cells. Alternatively, the immunophenotype of ASCs permits sorting using a flow cytometry-based cell sorter. As a result of the separation step or cell sorting, a population of enriched ASCs can be obtained. Preferably, the population of ASCs is a purified cell population. The isolated ASCs can then be cultured and expanded in vitro using methods disclosed herein or conventional methods.

A medium useful for culturing ASCs is referred to herein as “stromal cell medium”. Any medium capable of supporting fibroblasts in cell culture may be used as a stromal cell medium. Media formulations that support the growth of fibroblasts include, but are not limited to, Minimum Essential Medium Eagle, ADC-1, LPM (bovine serum albumin-free), F10 (HAM), F12 (HAM), DCCM1, DCCM2, RPMI 1640, BGJ Medium (with and without Fitton-Jackson Modification), Basal Medium Eagle (BME-with the addition of Earle's salt base), Dulbecco's Modified Eagle Medium (DMEM-without serum), Yamane, IMEM-20, Glasgow Modification Eagle Medium (GMEM), Leibovitz L-15 Medium, McCoy's 5A Medium, Medium M199 (M199E-with Earle's salt base), Medium M199 (M199H-with Hank's salt base), Minimum Essential Medium Eagle (MEM-E-with Earle's salt base), Minimum Essential Medium Eagle (MEM-H-with Hank's salt base) and Minimum Essential Medium Eagle (MEM-NAA with nonessential amino acids), and the like. A preferred medium for culturing ASCs is DMEM, more preferably DMEM/F12 (1:1).

Additional non-limiting examples of media useful in the methods of the invention may contain fetal serum of bovine or other species at a concentration at least 1% to about 30%, preferably at least about 5% to 15%, most preferably about 10%. Embryonic extract of chicken or other species can be present at a concentration of about 1% to 30%, preferably at least about 5% to 15%, most preferably about 10%.

An example of a stromal cell medium is a medium comprising DMEM/F 12 Ham's, 10% fetal bovine serum (FBS), 100 U penicillin/100 μg streptomycin (Pen-Strep) and 0.25 μg Fungizone® (generic name is amphotericin B). Typically, the stromal cell medium comprises a base medium, serum and an antibiotic/antimycotic. However, ASCs may be cultured in stromal cell medium without an antibiotic/antimycotic and supplemented with at least one growth factor. Preferably the growth factor is human epidermal growth factor (hEGF). The preferred concentration of hEGF is about 1-50 ng/ml, more preferably the concentration is about 5 ng/ml. The preferred base medium is DMEM/F12 (1:1). The preferred serum is fetal bovine serum (FBS) but other sera may be used, including horse serum or human serum. Preferably up to 20% FBS will be added to the above medium in order to support the growth of stromal cells. However, a defined medium can be used if the necessary growth factors, cytokines, and hormones in FBS for stromal cell growth are identified and provided at appropriate concentrations in the growth medium. It is further recognized that additional components may be added to the culture medium. Such components include, but are not limited to, antibiotics, antimycotics, albumin, growth factors, amino acids, and other components known to the art for the culture of cells. Antibiotics which can be added into the medium include, but are not limited to, penicillin and streptomycin. The concentration of penicillin in the culture medium is about 10 to about 200 units per ml. The concentration of streptomycin in the culture medium is about 10 to about 200 μg/ml. However, the invention should in no way be construed to be limited to any one medium for culturing stromal cells. Rather, any media capable of supporting stromal cells in tissue culture may be used.

Following isolation, ASCs are incubated in stromal cell medium, in a culture apparatus for a period of time or until the cells reach confluency before passing the cells to another culture apparatus. Following the initial plating, the cells can be maintained in culture for a period of about 6 days to yield the Passage 0 (P0) population. The cells may be passaged for an indefinite number of times, each passage comprising culturing the cells for about 6-7 days, during which time the cell doubling time can range between about 3 to about 5 days. The culturing apparatus can be of any culture apparatus commonly used in culturing cells in vitro. A preferred culture apparatus is a culture flask, with a more preferred culture apparatus being a T-225 culture flask.

ASCs may be cultured in stromal cell medium supplemented with hEGF in the absence of an antibiotic/antimycotic for a period of time or until the cells reach a certain level of confluence. Preferably, the level of confluence is greater than 70%. More preferably, the level of confluence is greater than 90%. A period of time can be any time suitable for the culture of cells in vitro. Stromal cell medium may be replaced during the culture of ASCs at any time. Preferably, the stromal cell medium is replaced every 3 to 4 days. ASCs are then harvested from the culture apparatus whereupon they may be used immediately or cryopreserved to be stored for use at a later time. ASCs may be harvested by trypsinization, EDTA treatment, or any other procedure used to harvest cells from a culture apparatus.

ASCs described herein may be cryopreserved according to routine procedures. Preferably, about one to ten million cells are cryopreserved in stromal cell medium containing 10% DMSO in vapor phase of liquid N₂. Frozen cells may be thawed by swirling in a 37° C. bath, resuspended in fresh growth medium, and expanded as described above.

The immunophenotype and immunogenic properties of ASCs are defined as a function of culturing procedures (i.e. adherence property, passage number, length of time in culture). Freshly isolated stromal vascular fraction (SVF) cells and early passaged ASCs stimulate peripheral blood mononuclear cells (PBMCs), whereas later passaged ASCs cells do not, indicating significantly reduced or null immunogenicity. Specifically, human SVF cells and early-passaged adherent cells derived from adipose tissue elicit a dose-dependent mixed lymphocyte reaction (MLR) response comparable to that of allogeneic PMBCs. With progressive passaging, ASCs elicit a much-decreased MLR response. By Passage 1 (P1), the MLR response elicited by ASCs is comparable to that observed with autologous PBMCs. Without being bound by theory, it is believed that the reduced immune resonse is due to the lack of expression of Class II Major Histocompatibility Antigens (MHA). Data also support that later-passaged cells may express immunosuppressive factors inhibiting the proliferative response of PBMCs to known stimulator cells.

The observed lack of immunogenic characteristics of late passaged ASCs is a robust predictor of a reduced likelihood of an immune rejection by either the host or the graft with respect to administering a composition of the invention to a mammal to alleviate or treat a tissue defect. For use in the compositions and methods of the instant invention, most particularly in allogeneic applications, non-immunogenic ASCs are preferred. Thus, ASCs at P1 or later are preferred in the compositions and methods of the invention, more preferred is ASCs at P2, still more preferred is ASCs at P3 and most preferred is ASCs passaged at least to P4.

As encompassed in the present invention, ASCs are typically isolated from liposuction material from a human. If the composition of the present invention is to be implanted into a human subject, it is preferable that the ASCs be isolated from that same subject so as to provide for an autologous graft or, if the intended recipient has an identical twin, a syngeneic graft is also preferred. However, advantageously, allogeneic grafts are also possible given the significantly reduced immunogenicity of later-passaged ASCs. This is particularly advantageous because it enables graft implants in emergency or otherwise time-critical situations with a significantly reduced likelihood of host immune rejection of the graft or GVHD. Xenogeneic grafts are also contemplated in the methods of the instant invention.

Genetically modified ASCs are also useful in the instant invention. Genetic modification may, for instance, result in the expression of exogenous genes (“transgenes”) or in a change of expression of an endogenous gene. Such genetic modification may have therapeutic benefit. Alternatively, the genetic modification may provide a means to track or identify the so-modified cells, for instance, after implantation of a composition of the invention into an individual. Tracking a cell may include tracking migration, assimilation and survival of a transplanted genetically-modified cell. Genetic modification may also include at least a second gene. A second gene may encode, for instance, a selectable antibiotic-resistance gene or another selectable marker.

Proteins useful for tracking a cell include, but are not limited to, green fluorescent protein (GFP), any of the other fluorescent proteins (e.g., enhanced green, cyan, yellow, blue and red fluorescent proteins; Clontech, Palo Alto, Calif.), or other tag proteins (e.g., LacZ, FLAG-tag, Myc, His₆, and the like).

When the purpose of genetic modification of the cell is for the production of a biologically active substance, the substance will generally be one that is useful for the treatment of a given disorder. For example, it may be desired to genetically modify cells so that they secrete a certain growth factor product associated with bone or soft tissue formation. Growth factor products to induce growth of other, endogenous cell types relevant to tissue repair are also useful. For instance, growth factors to stimulate endogenous capillary and/or microvascular endothelial cells can be useful in repair of soft tissue defect, especially for larger volume defects.

The cells of the present invention can be genetically modified by having exogenous genetic material introduced into the cells, to produce a molecule such as a trophic factor, a growth factor, a cytokine, and the like, which is beneficial to culturing the cells. In addition, by having the cells genetically modified to produce such a molecule, the cell can provide an additional therapeutic effect to the mammal when transplanted into a mammal in need thereof. For example, the genetically modified cell can secrete a molecule that is beneficial to cells neighboring the transplant site in the mammal.

As used herein, the term “growth factor product” refers to a protein, peptide, mitogen, or other molecule having a growth, proliferative, differentiative, or trophic effect on a cell. For example, growth factor products useful in the treatment of bone disorders include, but are not limited to, FGF, TGF-β, insulin-like growth factor, and bone morphogenetic protein (BMP).

The ASCs may be genetically modified using any method known to the skilled artisan. See, for instance, Sambrook et al. (2001, Molecular Cloning: A Laboratory Manual, Cold Spring Harbor Laboratory Press, Cold Spring Harbor, N.Y.), and in Ausubel et al,. Eds, (1997, Current Protocols in Molecular Biology, John Wiley & Sons, New York, N.Y.). For example, an ASC may be exposed to an expression vector comprising a nucleic acid including a transgene, such that the nucleic acid is introduced into the cell under conditions appropriate for the transgene to be expressed within the cell. The transgene generally is an expression cassette, including a polynucleotide operably linked to a suitable promoter. The polynucleotide can encode a protein, or it can encode biologically active RNA (e.g., antisense RNA or a ribozyme). Thus, for example, the polynucleotide can encode a gene conferring resistance to a toxin, a hormone (such as peptide growth hormones, hormone releasing factors, sex hormones, adrenocorticotrophic hormones, cytokines (e.g., interfering, interleukins, lymphokines), etc.), a cell-surface-bound intracellular signaling moiety (e.g., cell adhesion molecules, hormone receptors, etc.), a factor promoting a given lineage of differentiation (e.g., bone morphogenic protein (BMP)), etc.

Within the expression cassette, the coding polynucleotide is operably linked to a suitable promoter. Examples of suitable promoters include prokaryotic promoters and viral promoters (e.g., retroviral ITRs, LTRs, immediate early viral promoters (IEp), such as herpesvirus IEp (e.g., ICP4-IEp and ICP0-IEEp), cytomegalovirus (CMV) IEp, and other viral promoters, such as Rous Sarcoma Virus (RSV) promoters, and Murine Leukemia Virus (MLV) promoters). Other suitable promoters are eukaryotic promoters, such as enhancers (e.g., the rabbit .beta.-globin regulatory elements), constitutively active promoters (e.g., the .beta.-actin promoter, etc.), signal specific promoters (e.g., inducible promoters such as a promoter responsive to RU486, etc.), and tissue-specific promoters. It is well within the skill of the art to select a promoter suitable for driving gene expression in a predefined cellular context. The expression cassette can include more than one coding polynucleotide, and it can include other elements (e.g., polyadenylation sequences, sequences encoding a membrane-insertion signal or a secretion leader, ribosome entry sequences, transcriptional regulatory elements (e.g., enhancers, silencers, etc.), and the like), as desired.

The expression cassette containing the transgene should be incorporated into a genetic vector suitable for delivering the transgene to the cells. Depending on the desired end application, any such vector can be so employed to genetically modify the cells (e.g., plasmids, naked DNA, viruses such as adenovirus, adeno-associated virus, herpesviruses, lentiviruses, papillomaviruses, retroviruses, etc.). Any method of constructing the desired expression cassette within such vectors can be employed, many of which are well known in the art (e.g., direct cloning, homologous recombination, etc.). The choice of vector will largely determine the method used to introduce the vector into the cells (e.g., by protoplast fusion, calcium-phosphate precipitation, gene gun, electroporation, DEAE dextran or lipid carrier mediated transfection, infection with viral vectors, etc.), which are generally known in the art.

II. Differentiation of Adult Stem Cells

ASCs may be differentiated into a number of different lineages, including adipocyte, chondrocyte, endothelial, hematopoietic support, hepatocyte, neuronal, myogenic and osteoblast lineages. Methods for inducing lineage-specific differentiation are known to the skilled artisan. Methods to characterize differentiated cells that develop from the ASCs include, but are not limited to, histological, morphological, biochemical and immunohistochemical methods, or using cell surface markers, or genetically or molecularly, or by identifying factors secreted by the differentiated cell, and by the inductive qualities of the differentiated ASCs.

A preferred method of inducing adipogenesis in ASCs involves culturing cells in adipogenic medium. An exemplary adipogenic medium is DMEM supplemented with ITS+3 (SigmaAldrich, St. Louis, Mo.), Pen-Strep, fungizone, 0.1 mM nonessential amino acids and adipogenic stimulants (AD). ITS+3 is insulin (10 mg/liter), transferrin (5.5 mg/liter), selenium (5 μg/liter), bovine serum albumin (0.5 mg/liter), linoleic acid (4.7 μg/liter), and oleic acid (4.7 μg/liter). Exemplary adipogenic stimulants include 0.5 mM 3-isobutyl-1-methyl-xanthine, 1 μM dexamethasone, 5 μg/ml insulin, and 50 μM indomethacin.

A preferred method of inducing osteogenesis in ASCs involves culturing cells in osteogenic medium. An exemplary osteogenic medium is DMEM supplemented with ITS+3, Pen-Strep and Fungizone®, and osteogenic stimulants (50 μg/ml ascorbic acid-2-phosphate, 10 nM dexamethasone, 7 mM beta-glycerophosphate and 1 μg/ml BMP-2). Alternatively, the BMP-2 may be provided by the biocompatible scaffold. In this embodiment, the BMP-2 is typically omitted from the osteogenic medium.

A preferred method of inducing chondrogenesis involves culturing cells in chondrogenic medium. An exemplary chondrogenic medium is DMEM supplemented with ITS+3, Pen-Strep and Fungizon®e, 0.1 mM nonessential amino acids, and chondrogenic stimulants consisting of 50 μg/ml ascorbic acid-2-phosphate, 10 nM dexamethasone, 5 μg/ml insulin and 5 ng/ml TGF β1.

While the culturing conditions are not considered limiting, samples are incubated, typically in a humidified incubator at 37° C. and 5% CO₂. Half of the medium is replaced about every 2-3 days for the duration of the culturing period. The length of time of culturing may be several weeks.

Differentiation may be induced in vitro in appropriate culture conditions or may be induced in vivo. In vivo differentiation may rely solely on endogenous differentiation cell signals and factors or may be supplemented by exogenous differentiation cell signals and factors. Exogenous factors may be provided in any appropriate manner, including, but not limited to, genetically-modified adult stem cells expressing one or more factors, scaffolds covalently or non-covalently modified with factors, systemic administration and localized administration at the site of the graft implant.

III. Preparing Biocompatible Scaffolds

Scaffolds for use in the instant invention are made from biocompatible materials. Non-limiting examples of biocompatible materials include silk, collagen, and other protein-based polymers. The ideal properties of the biocompatible materials for use in the instant invention include: mechanical integrity, thermal stability, ability to self-assemble, non-immunogenic, bioresorbable, slow degradation rate, capacity to be functionalized with, for instance, cell growth factors, and plasticity in terms of processing into different structural formats. Silk provides most, if not all, of these properties and is therefore the preferred biocompatible material for the compositions of the instant invention.

Scaffolds for use in the instant invention may be any structural format, including nanoscale diameter fibers from electrospinning, fiber bundles and films. Methods of forming these various formats from silk are known to the skilled artisan. See, for instance, Jin et al., 2002, Biomacromolecules 3:1233-1239; Jin et al., 2004, Biomacromolecules 5:711-717; Altman et al., 2002, Biomaterials 23:4131-4141; Altman et al., 2002, J Biomech. Eng. 124:742-749; and Meinel et al., 2004, Biotechnol. Bioeng. 88:379-391, and U.S. Patent Publication No. 20050260706, each incorporated herein by reference in its entirety. Preferably, the scaffolds for use in the instant invention are 3-dimensional matrices. Methods of making 3-dimensionsal silk matrices are known to the skilled artisan. See, for instance, Kim et al., 2005, Biomaterials 26:2775-2785, Nazarov et al., 2004, Biomacromolecules 5:718-726, and U.S. Patent Publication No. 2004062697, each incorporated herein by reference in its entirety. Silk hydrogels may also be useful as temporary matrices for filling in defects during osseo-integration and as well as for soft tissue reconstruction, such as contour reconstruction for orofacial and cranial tissues. Methods of making silk hydrogels are known to the skilled artisan. See, for instance, Kim et al., 2004, Biomacromolecules 5:786-792 and Jin et al., 2003, Nature 424: 1057-1061, each incorporated herein by reference in its entirety.

There are numerous ways known to the skilled artisan for making porous silk scaffolds, including freeze-drying, salt leaching and gas foaming (Nazarov et al, 2004, Biomacromolecules 5:718-726, incorporated herein by reference in its entirety). When the desired scaffold properties are high porosity and very high compressive strength, gas foaming may be preferred. When the desired scaffold properties are high porosity and low compressive strength, the freeze-dried scaffolds may be preferred. For the scaffolds used in alleviating or treating a bone defect, the preferred method of making the silk scaffold is salt leaching. The salt leaching method is preferably an all-aqueous method when avoidance of organic solvents is necessary. Salt leaching methods yield scaffolds having high porosity and high compressive strength. Preferred are silk scaffolds having a porosity of at least 90% and more preferably, at least about 93%. The pores are preferably homogenous and interconnected. Pore size in the silk scaffold is determined by the size of the salt particles used in the salt leaching process. Larger salt particles yield larger pores in the silk scaffold. Preferably the pores are about 50 to about 1200 microns, more preferably about 250 to about 1100 microns and more preferably about 450 to about 1000 microns. Preferred compressive strength for the silk scaffold is at least about 250 KPa, more preferably at least about 300 KPa and more preferably about 320 KPa. Preferred modulus is about 2800 to about 4000 KPa, more preferably about 3000 to about 3750 KPa and most preferably about 3200 to about 3500 KPa. A preferred method of making a silk scaffold by a salt leaching method is presented in the Examples. Scaffolds may be sterilized by autoclaving them, treatment with ethylene oxide gas or with alcohol.

The scaffolds of the instant invention may be modified with one or more molecules. Any molecule may be attached, covalently or non-covalently, to the biomaterial to modify it. For instance, cell growth factors may be covalently bound to the scaffold material. Silk, the preferred biocompatible material for the scaffold, is readily functionalized, using well-known amino acid side chain chemistries such as carboiimide chemistry, thus allowing covalent modification (see, for instance, Sofia et al., 2001, J Biomed. Mater. Res. 54:139-148 and Karageorgiou et al., 2005, J Biomed. Mater. Res. 71:528-537, each incorporated herein by reference in its entirety). Alternatively, a matrix may be coated with a molecule. Molecules for modification are preferably non-immunogenic in the intended recipient individual. A molecule whose sequence is native to the intended recipient individual is considered to be non-immunogenic. Preferred molecules for modification are molecules that function in controlling cell attachment, cell differentiation and cell signaling. Non-limiting examples of such molecules include the integrin binding tripeptide RGD, parathyroid hormone (PTH) and BMP-2. In some embodiments, a silk scaffold is covalently modified with RGD to a final density of about 3 to about 4 pM/cm². In some embodiments, a silk scaffold is non-covalently modified with BMP, preferably BMP-2, at about 2 to about 3 micrograms/scaffold for a scaffold having dimensions of about 5 mm diameter and 2 mm thickness. Larger or smaller scaffolds may have more or less BMP, respectively.

An exemplary method of preparing a 3-dimensional silk scaffold covalently modified with RGD is as follows. B. mori cocoons are boiled for 30 minutes in an aqueous solution of 0.02 M Na₂CO₃, then rinsed thoroughly with water to remove the undesirable, glue-like sericin proteins. The extracted silk is then dissolved in 9.3 M LiBr solution at room temperature, to yield, for instance, a 5% (w/v) solution. This solution is dialyzed in water and then in a coupling buffer (0.1M N-morpholinoethane sulfonic acid (MES) and 0.5 M NaCl, pH=6). Fifty ml from this solution is activated with 1.02 g 1-ethyl-3-(3-dimethylaminopropyl)-carboiimide (EDC) and 1.53 g N-hydrozysuccinimide (NHS) for 15 minutes. The activation reaction is stopped by the addition of 3.6 ml beta-mercaptoethanol. Immediately 25 mg of RGD is added and the coupling reaction is allowed to proceed for about 2 hrs. The coupling reaction is quenched by the addition of 10 mM hydroxylamine hydrochloride. The resulting solution of RGD covalently coupled to silk (silk-RGD) is dialyzed against water and lyophilized.

To prepare scaffolds, lyophilized silk-RGD is dissolved in hexafluorisopropanol (HFIP) at high concentration (>16% w/v) and loaded into molds containing a salt porogen, for instance, NaCl. In one embodiment, the molds are about 5 mm in diameter and at least 2 mm thick and the resulting scaffold is about 5 mm in diameter and about 2 mm thick. The filled molds are then dried by evaporating the solvent in a fume hood at room temperature. Methanol is used to convert the silk protein to a crystalline state (silk II). Pore sizes are determined by the particle size of the porogen. In some embodiments, the pore size range is 450-550 microns. The salt porogen is removed in cold water for 48 hours with four exchanges each day to assure complete removal. Characterization of structure (FTIR) and morphology (SEM) may be conducted on the scaffolds.

As described elsewhere herein, scaffolds may be additionally or alternatively modified non-covalently. An exemplary method of non-covalently modifying a covalently-modified scaffold is as follows.

BMP-2 is dialyzed to remove formulation buffer components and is then sterilized, preferably filter sterilized using 0.22 micron syringe filters. Autoclaved silk-RGD scaffolds are placed in a container, for instance a 10 ml syringe, and contacted for 6 hours with 1.5 ml of 0.05 mg/ml BMP-2 in Dulbecco's phosphate buffered saline solution. The scaffolds are then rinsed with sterile saline solution to remove unbound protein and are seeded with cells. Loading and release profiles of BMP-2 may be checked with radioactive (I¹²⁵-BMP-2).

ASCs are seeded onto sterile scaffolds using aseptic techniques. In one embodiment, the sterile scaffolds are prewetted prior to seeding by overnight incubation in DMEM or other suitable medium. In a preferred embodiment, seeding is done with P2 ASCs, more preferably P3 ASCs and even more preferably ASCs at least at P4. For scaffolds approximately 5 mm in diameter and 2 mm thick, cells are seeded at about 5×10⁵ to 5×10⁶ cells per scaffold. The number of cells seeded on a scaffold may be adjusted based on the size of the scaffold. The skilled artisan is able to determine the optimal number of cells to seed on any particular size and shape scaffold and intended application. The number of adult stem cells seeded onto a scaffold is, however, not viewed as limiting. Without being bound by theory, seeding the scaffold with a high density of adult stem cells may accelerate tissue generation.

An exemplary method of seeding a scaffold is to suspend cells in DMEM supplemented with ITS+3, Pen-Strep and Fungizone® and an aliquot is applied to each face of a scaffold. In one embodiment, about 3×10⁶ cells are suspended in 200 microliters of DMEM supplemented with ITS+3, Pen-Strep and Fungizone® and are seeded on a scaffold in 2-100 microliter aliquots. In another exemplary method, cells are suspended in Matrigel® and kept on ice to prevent gelation prior to applying the suspension to a scaffold.

ASCs may be differentiated prior to seeding them onto scaffolds, after seeding and prior to implantation, or after seeding and after implantation. In a preferred embodiment, ASCs are differentiated after seeding and prior to transplantation, as described in the Examples. This embodiment leads to faster and more mature tissue regeneration.

Any culture methods may be used for expanding and/or differentiating cells seeded onto silk scaffolds, including static, 2-dimensional culture methods. However, dynamic 3-dimensional culture conditions are preferred for enhanced cell viability and adipogenic or osteogenic differentiation.

Dynamic, 3-dimensional culturing may be performed by means of the spinner flask system (e.g., Meinel et al., 2004, Ann. Biomed. Eng. 32:112-122, incorporated herein by reference in its entirety). Spinner flask systems are advantageous in providing a larger number of scaffolds to be processed simultaneously and provide more consistent outcomes in culturing. In an exemplary method of spinner flask culturing, P2 ASCs are applied to silk scaffolds at 3×10⁶ cells/scaffold. The medium includes 10% fetal calf serum (FCS) supplemented with the serum supplement ITS+3 (Sigma-Aldrich, St Louis, Mo.) in order to eliminate potential immune responses from bovine serum following implantation into a non-bovine hosts. The cells are suspended in 200 μl of DMEM supplemented with ITS+3, Pen-Strep and Fungizone® and 100 μl of the resulting cell solution is applied to each face of a scaffold. The scaffolds are subsequently incubated with the concentrated cell solution for 3 hours in a humidified incubator at 37° C./5% CO₂ with intermittent agitation every 30 minutes to promote residual cell attachment while being maintained. The scaffolds are then incubated under the same conditions for an additional 12 hours in the above medium (DMEM supplemented with ITS+3, Pen-Strep and Fungizone®) prior to transfer within the spinner flask. The seeded scaffolds are threaded onto needles embedded in the stoppers of the spinner flask (two scaffolds per needle on four needles per flask) as previously described (Meinel et al., 2004, Ann, Biomed. Eng. 32:112-122).

For osteogenesis, flasks are filled with 120 ml osteogenic medium (DMEM supplemented with ITS+3, Pen-Strep and Fungizone®, and osteogenic stimulants consisting of 50 μg/ml ascorbic acid-2-phosphate, 10 nM dexamethasone, 7 mM beta-glycerophosphate). For adipogenesis, flasks are filled with 120 ml of adipogenic medium (DMEM supplemented with ITS+3, Pen-Strep and Fungizone®, 0.1 mM nonessential amino acids and adipogenic stimulants (AD) consisting of 0.5 mM 3-isobutyl-1-methyl-xanthine, 1 μM dexamethasone, 5 μg/ml insulin, and 50 μM indomethacin).

Flasks are placed in a humidified incubator at 37° C./5% CO₂, with the side arm caps loosened to permit gas exchange, and are stirred with a magnetic bar at 50 rpm. Medium is replaced at a rate of 50% every 2-3 days for the duration of the culturing. Samples are cultivated for about 4 weeks.

IV. Tissue Regeneration Applications

The objective of the tissue regeneration therapy approach is to deliver high densities of repair-competent cells (or cells that can become competent when influenced by the local environment) to the defect site in a format that optimizes both initial wound mechanics and eventual neotissue production. The composition of the instant invention is particularly useful in methods to alleviate or treat bone defects or soft tissue defects in individuals. Advantageously, the composition of the invention provides for improved bone regeneration or soft tissue regeneration. Specifically, the tissue regeneration is achieved more rapidly as a result of the inventive composition.

The composition of the invention may be administered to an individual in need thereof in a wide variety of ways. Preferred modes of administration include intravenous, intravascular, intramuscular, subcutaneous, intracerebral, intraperitoneal, soft tissue injection, surgical placement, arthroscopic placement, and percutaneous insertion, e.g. direct injection, cannulation or catheterization. Most preferred methods result in localized administration of the inventive composition to the site or sites of tissue defect. Any administration may be a single application of a composition of invention or multiple applications. Administrations may be to single site or to more than one site in the individual to be treated. Multiple administrations may occur essentially at the same time or separated in time.

There are numerous bone defects for which the inventive method is applicable. Such defects include, but are not limited to, segmental bone defects, non-unions, malunions or delayed unions, cysts, tumors, necroses or developmental abnormalities. Other conditions requiring bone augmentation, such as joint reconstruction, cosmetic reconstruction or bone fusion, such as spinal fusion or joint fusion, are treated in an individual by administering, for example, into the site of the bone defect, a composition of the invention to an extent sufficient to augment bone formation therefrom, thereby alleviating or treating the defect. The composition can also contain one or more other components which degrade, resorb or remodel at rates approximating the formation of new tissue. In a typical application, the composition is inserted in the defect and results in osteogenic healing of the defect. Preferably the composition for use in treating a bone defect comprises P2 ASCs, more preferably human P2 ASCs, seeded on a silk scaffold modified with at least one of RGD and BMP-2 and cultured in vitro in osteogenic medium in dynamic, 3-dimensional culture conditions, as described elsewhere herein. In one embodiment, the composition is an autologous graft. In another embodiment, the composition is an allogeneic graft.

Numerous soft tissue defects may also be alleviated or treated using the compositions and methods of the invention. Non-limiting examples of soft tissue reconstruction include breast reconstruction after masectomy, breast augmentation, and soft tissue reconstruction after tumor resection, such as facial tissue. A composition of the invention is administered to an extent sufficient to achieve alleviation or treatment of the soft tissue defect. Advantageously, the composition and method of the invention improve on prior art methods of soft tissue defect in reducing the extent of undesirable outcomes, such as dimpling. Preferably the composition for use in treating a soft tissue defect comprises P2 ASCs, more preferably P2 human ASCs, seeded on a silk scaffold and cultured in vitro in adipogenic medium in dynamic, 3-dimensional culture conditions, as described elsewhere herein. In one embodiment, the composition is an autologous graft. In another embodiment, the composition is an allogeneic graft.

### EXPERIMENTAL EXAMPLES

The invention is further described in detail by reference to the following experimental examples. These examples are provided for purposes of illustration only, and are not intended to be limiting unless otherwise specified. Thus, the invention should in no way be construed as being limited to the following examples, but rather, should be construed to encompass any and all variations which become evident as a result of the teaching provided herein.

The materials and methods used in the experiments presented in the Experimental Examples below are now described.

### Example 1

### Isolation and Charaterization of Adipose-Derived Stem Cells (ASCs)

Adipose-derived stem cells were isolated by subjecting a sample of adipose tissue from a human liposuction specimen to collagenase digestion, differential centrifugation and then expansion in culture as previously described (Aust et al., 2004, Cytotherapy 6:7-14; Halvorsen et al., 2001, Metabolism 50: 407-413; Sen et al., 2001, J Cell Biochem. 81: 312-319; Gimble et al., 2003, Cytotherapy 5:362-369). A single gram of tissue typically yields between 50,000 to 100,000 stromal cells within about 24 hours of culture using this method, and a mean of about 250,000 cells within 6 days of culture. Using this method, it is possible to produce in excess of 500 million cells within a 2 week period after a standard lipoaspirate.

Passage 2 (P2) ASCs thus isolated were characterized with respect to their cell surface markers and their differentiation potential. As shown in Table 1, the ASCs exhibited an immunophenotype and differentiation potential comparable to bone marrow-derived mesenchymal stem cells (BMSCs).

TABLE 1
Characterization of Passage 2 Human ASCs

| | | Differentiation |
|---|---|---|
| Surface Positive Markers | Surface Negative Markers | Potential |
| CD9, CD10, CD13, CD29, | CD11, CD14, CD16, | Adipocyte |
| CD44, CD49d, CD54, | CD18, CD31, CD45, | Chondrocyte |
| CD55, CD59, CD71, | CD50, CD56, CD62, | Hematopoietic |
| CD73, CD90, CD105, | CD104, Factor VIII | Support |
| CD106, CD146, CD166, α- | related Ag, HLA-DR | Myocyte |
| smooth muscle actin, | | (Cardiac, |
| collagen type I, collagen | | Skeletal) |
| type III, HLA-ABC, | | Myofibroblast |
| nestin, osteopontin, | | Neuronal |
| steonectin, vimentin | | Osteoblast |

The surface immunotype of ASCs was also characterized during the isolation and expansion process using flow cytometric analysis. In the stromal vascular fraction (SVF), 10% of the cells express the antigen CD45, a unique protein marker of hematopoietic cells. Additionally, 6.1% of the cells express CD14, the endotoxin receptor associated specifically with macrophages and monocytes. With further expansion and passage, there was a progressive loss of these hematopoietic cells in the culture. Specifically, passage and expansion of the ASCs was accompanied by increased expression of stromal-associated cell surface adhesion proteins, such as integrin β1 (CD29) and activated lymphocyte cell adhesion molecule (CD166), and receptors for hyaluronic acid (CD44) and transforming growth factor β (endoglin, CD105). Two stem cell-associated markers are of particular interest: CD34 and aldehyde dehydrogenase (ALDH) have been used to define and isolate hematopoietic stem cells. In the ASC cultures, both markers increased to peak levels at passages P0 or P1; while ALDH was sustained through further passage, CD34 declined after continued expansion.

Using 2-dimensional gel electrophoresis/tandem mass spectroscopy, the proteome of ASCs was further characterized (Delany et al., 2005, Mol. Cell Proteomics 4:731-740; incorporated herein by reference in its entirety). Protein lysates obtained from four individual donors were compared before and after adipocyte differentiation by two-dimensional gel electrophoresis and tandem mass spectroscopy. Over 170 individual protein features in the undifferentiated adipose-derived adult stem cells were identified. Following adipogenesis, over 40 proteins were up-regulated by greater than or equal to 2-fold, whereas 13 showed a greater than or equal to 3-fold reduction. The majority of the modulated proteins belonged to the following functional categories: cytoskeleton, metabolic, redox, protein degradation, and heat shock protein/chaperones. Additional immunoblot analysis documented the induction of four individual heat shock proteins and confirmed the presence of the heat shock protein 27 phosphoserine 82 isoform, as predicted by the proteomic analysis, as well as the crystallin alpha phosphorylated isoforms.

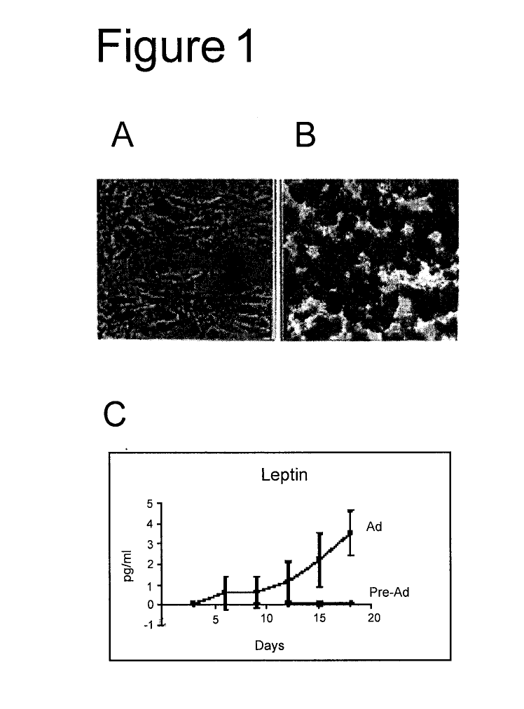

The adipogenic potential of ASCs was characterized. In the presence of dexamethasone, insulin, isobutylmethylxanthine and a thiazolidinedione, ASCs undergo adipogenesis adipogenesis. The image in FIG. 1A shows ASCs that have not undergone adipogenesis. FIG. 1B shows ASCs that have undergone adipogenesis. ASCs that have undergone adipogenesis accumulated lipid vacuoles which can be stained for neutral lipid with a dye called Oil Red O. They also expressed adipocyte-specific markers, including the secreted cytokine leptin (FIG. 1C) and the fatty acid binding protein aP2. The cells displayed a lipolytic response to adrenergic compounds, a biochemical characteristic of mature primary adipocytes.

Colony forming unit assays use limit dilution methods to quantify the frequency of specific lineage progenitors. The stromal vascular fraction, which contained a mean number of 308,849±140,354 nucleated cells per ml of lipoaspirate (mean±S.D., n=14 donors), was serially diluted by 2-fold dilutions in 96 well plates at concentrations of 10⁴ to 4 cells per well. After 9 days in the culture, the number of wells containing cell colonies staining positive for toluidine blue or alkaline phosphatase was used to determine the frequency of CFU-F and CFU-ALP, respectively. At that time, identical plates were induced to undergo adipogenesis and osteogenesis. The number of wells staining positive for neutral lipids by Oil Red O or for calcium phosphate by Alizarin Red was determined after an additional 9 days or >14 days, respectively. The resulting CFU frequencies are shown in Table 2. Values shown are mean±S.D. Further studies demonstrated that subsequent passage of the ASCs enriched the frequency of lineage specific CFUs by 3- to 10-fold.

TABLE 2

| CFU type | Frequency | |
|---|---|---|
| CFU-F | 1:32 ± 48 | (n = 12) |
| CFU-ALP | 1:328 ± 531 | (n = 12) |
| CFU-Ad | 1:28 ± 49 | (n = 10) |
| CFU-Ob | 1:16 ± 22 | (n = 7) |

Note:
F = fibroblast; ALP = alkaline phosphatase; Ad = adipocyte; Ob = osteoblast

A parallel approach was also used to assess the clonality of multipotent human ASCs. The SVF derived from three individual donors was plated at low density and colonies derived from single cells were ring cloned (Guilak et al., 2005, J Cell Physiol. July 14 Epub). Forty-five clones were expanded through four passages and then induced for adipogenesis, osteogenesis, chondrogenesis, and neurogenesis using lineage-specific differentiation media. Quantitative differentiation criteria for each lineage were determined using histological and biochemical analyses. Approximately 20% of the clones exhibited tripotency and over 30% were bipotent. By demonstrating the multipotentiality of the cells at a clonal level, these studies confirm the “stem cell” terminology used to describe ASCs.

### Example 2

### Immunogenicity of SVFs and Passaged ASCs

Mixed lymphocyte reactions (MLR) were used to assess the immunoregulatory effects of human adipose derived cells in vitro on a T-cell mediated immune response. The proliferation of peripheral blood mononuclear cells (PBMLs) was measured based on tritiated thymidine incorporation in the presence of increasing doses of irradiated stimulator cells. Three criteria were used in assessing the immunogenicity of cell populations. These were: 1) a statistically significant difference in the T cell proliferative response (CPM) relative to that induced by autologous peripheral blood mononuclear Cells (PBMCs) (p<0.05, Student's t test); 2) a difference of at least 750 CPM from the response induced to autologous PBMCs; and 3) a stimulation index (SI; CPM induced by the test population divided by CPM induced by autologous PBMCs) of at least 3.0. Autologous and allogeneic PBMLs served as negative and positive stimulator cell controls, respectively.

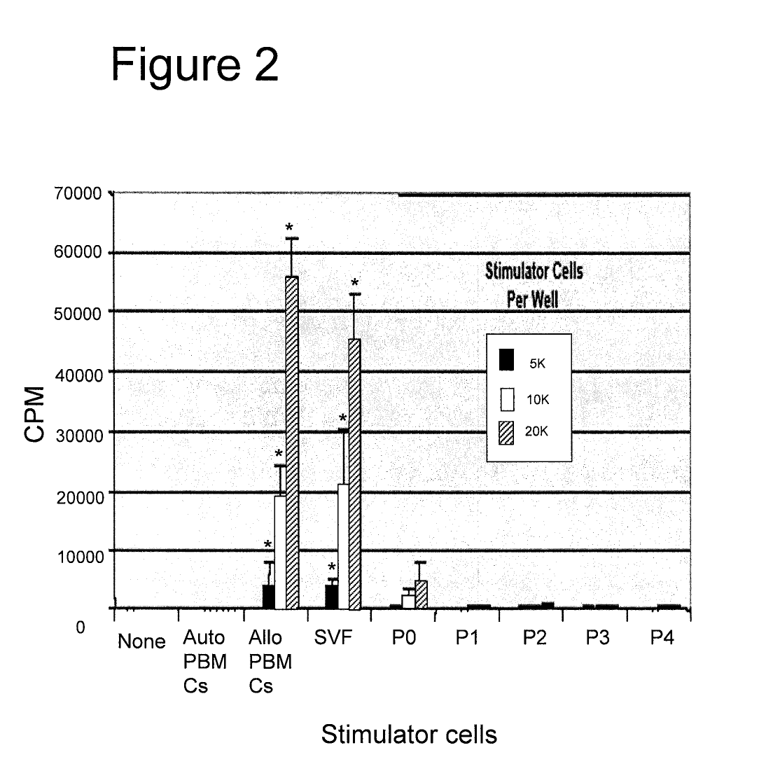

Representative data from a single donor is shown in FIG. 2. Human SVF cells elicited a dose-dependent MLR response comparable to that of allogeneic PBMLs. With progressive passage, the human ASCs elicited a decreased response that fell to undetectable levels by P1. Thus, the immunogenicity of ASCs significantly decreases as a function of adherence and length of time in culture.

In addition, the higher passage human ASCs displayed an inmunosuppressive effect. When added to MLRs in the presence of allogeneic PBMLs as stimulatory cells, the P2 and P3 human ASCs suppressed the proliferative response by 60-80% in a dose dependent manner. This compares favorably to fibroblast/stromal cells isolated from other tissue sites, including bone marrow, skin, connective tissue, fetal, lung, and spleen. The immunosuppressive effects of adipose-derived cells exceeded those of cells derived from each of the alternative sites. Similar findings regarding immunogenicity and immunosuppressive properties of BMSCs are known (Bartholomew et al., 2002, Exp. Hematol 30:42-48).

### Example 3

### Silk-Based Scaffolds

B. mori silk fibroin was prepared using a method that is a modification of earlier-reported procedures (e.g., Sofia et al., 2001, J Biomed. Mater. Res. 54:139-148, incorporated herein by reference in its entirety). Cocoons were boiled for 30 minutes in an aqueous solution of 0.02 M Na₂CO₃, then rinsed thoroughly with water to remove the undesirable, glue-like sericin proteins. The extracted silk was then dissolved in 9.3 M LiBr solution at room temperature yielding a 5% (w/v) solution. This solution was dialyzed in water using Slide-a-Lyzer® dialysis cassettes (Pierce Chemical Co, Rockford Ill.; MWCO 2000) and then lyophilized. The silk solution was prepared by dissolving lyophilized silk in hexafluoroisopropanol (HFIP) to a final 17% (w/v) concentration. Sodium chloride (NaCl) particles (NaCl particle size depend on the size of the desired pores, e.g., from 50 to 1,000 μm), acting as porogens, were added to Teflon disk-shaped molds and then the silk/HFIP solution was added. The weight ratio of porogen to silk was adjusted from 10:1 to 20:1 (salt to silk). The HFIP solvent in the mixture of silk/porogen was evaporated at room temperature, creating a silk/porogen composite. Immediately prior to exposure to water, the silk/porogen composite was immersed in methanol for 30 minutes to induce β-sheet structure and insolubility in aqueous solution (Nazarov et al., 2004, Biomacromolecules 5:718-726). Based on X-ray Photoelectron Spectroscopy (XPS) analysis as well as biological responses (hBMSC) to these scaffolds, HFIP and salt residual were not present.

In other strategies, variations on this approach have been developed to permit the formation of 3D silk fibroin scaffolds in an all aqueous process with similar porosities (>90%) and pore sizes (up to 1,000 microns) but with more rapid rates of degradation due to a lower content of beta sheet (Kim et al., 2005, Biomaterials 26:2775-2785, incorporated herein by reference in its entirety). The resultant 3D silk scaffolds have compressive strength and modulus up to 320±10 KPa and 3330±500 KPa, respectively, when formed from 10% aqueous solutions of fibroin. These data demonstrate that the fibroin scaffolds are mechanically robust in 3D format and meet or exceed mechanical properties of corresponding commonly used polymeric biomaterials (e.g., collagen, PLA). See Table 3.

TABLE 3

| | Compression | Compresssion |
|---|---|---|
| Material | Strength (KPa) | Modulus (KPa) |
| Silk-HFIP¹ | 175-250 | 450-1000 |
| Silk-water² | 320 | 3330 |
| PLA, PLGA, | 0.53 | 26-302 |
| PDLLA³ | | |
| Collagen⁴ | ˜15 | ˜150 |

¹HFIP-derived scaffolds, silkworm fibroin, gas foaming and salt leaching methods (Nazarov et al., 2004, Biomacromolecules 5: 718-726)
²Water-derived scaffolds, silkworm fibroin, salt leaching method (Kim et al., 2005, Biomaterials 26: 2775-2785)
³Poly(D,L-lactic-co-glycolic acid) by salt leaching, sintering (Nam et al., 2000, J. Biomed. Mater. Res. 53(1): 1-7; Hou et al., 2003, J. Biomed. Mater. Res. B Appl. Biomater. 67: 732-740)
⁴Collagen processed by lyophilization (Cho et al., 2001, Fibers Polym. 2: 64-70)

### Example 4

### Human BMSCs and Silk Scaffold Composition in Vitro for Bone-Like Tissue

Human BMSCs were isolated by density gradient centrifugation from whole bone marrow (25 cm³ harvests) obtained from Clonetics (Santa Rosa, Calif.). Briefly, samples of bone marrow were diluted in 100 ml of isolation medium (RPMI 1640 supplemented with 5% FBS). Bone marrow suspension in 20 ml aliquots was overlaid onto a poly-sucrose gradient (1,077 g/cm³, Histopaque®, Sigma, St. Louis, Mo.) and centrifuged at 800 g for 30 min at room temperature. The cell layer was carefully removed, washed in 10 ml isolation medium, pelleted and the contaminating red blood cells lysed in 5 ml of Pure-Gene® Lysis solution (Genta Systems, Minneapolis, Minn.). Cells were pelleted and suspended in expansion medium (DMEM, 10% FBS, 1 ng/ml bFGF) and seeded in 75 cm² flasks at a density of 5×10⁴ cells/cm². The adherent cells were allowed to reach approximately 80% confluence (12-17 days for the first passage). Adherent cells were trypsinized and replated every 6-8 days at about 80% confluence. The 2nd passage (P2) cells were usually used. Human BMSCs were characterized with respect to (a) the expression of surface antigens and (b) the ability to selectively differentiate into chondrogenic or osteogenic lineages in response to environmental stimuli.

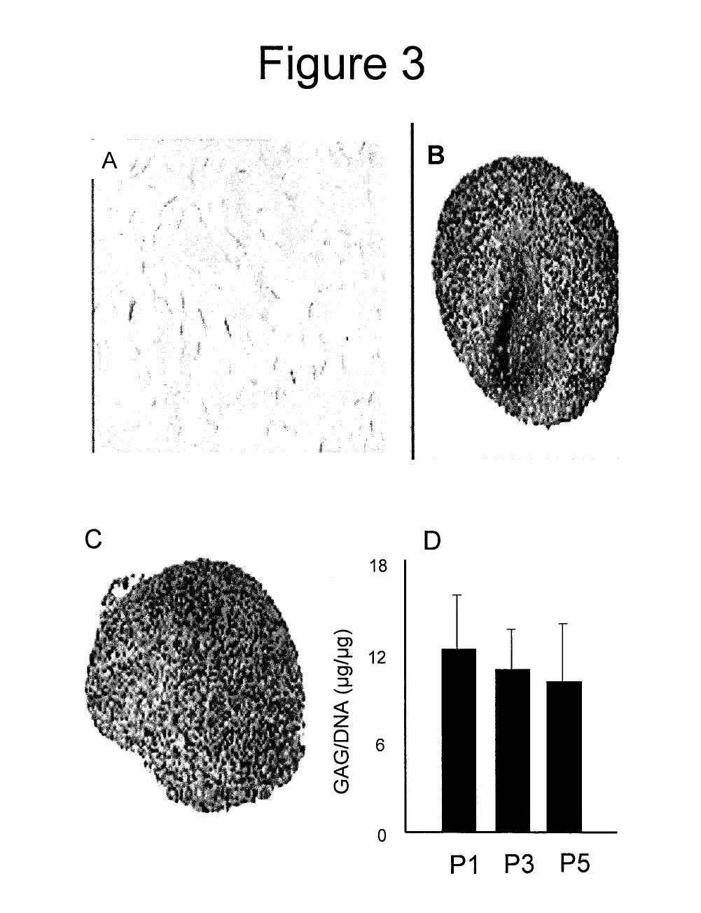

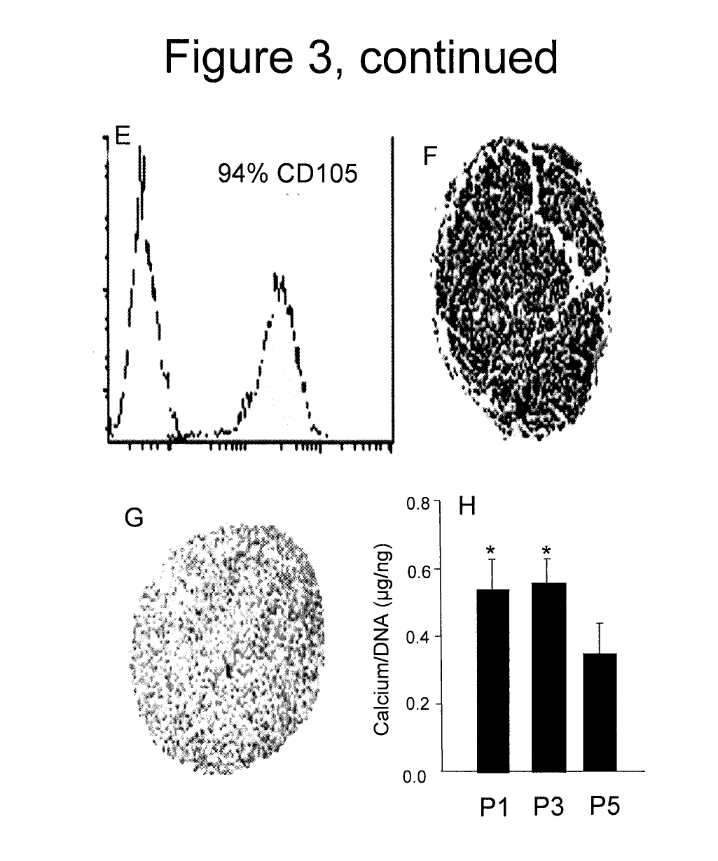

The expression of the following six surface antigens: CD44 (hyaluronate receptor), CD14 (lipopolysaccharide receptor), CD31 (PECAM-1/endothelial cells), CD34 (sialomucin/hematopoietic progenitors), CD71 (transferring receptor/proliferating cells), and CD105 (endoglin) was characterized by Fluorescence Activated Cell Sorting (FACS) analysis (Meinel et al., 2004, Biotechnol. Bioeng. 88:379-391; Meinel et al., 2004, J. Biomed.Mater. Res. A. 71:25-34). Cells were detached with 0.05% (w/v) trypsin, pelleted and resuspended at a concentration of 1×10⁷ cell/ml. Aliquots (50 μl) ofthe cell suspension were incubated for 30 minutes on ice with 2 μl of each of the following antibodies: anti-CD44 and anti-CD14 conjugated with fluoresceine isothiocyanate (CD44-FITC, CD 14-FITC), anti-CD31 conjugated with phycoerythrin (CD31-PE), anti CD34 conjugated with allophycocyanine (CD34-APC), anti CD71-APC, and anti-CD105 with a secondary rat-anti mouse IgG-FITC antibody (all antibodies from Neomarkers, Fremont Calif.). Cells were washed, suspended in 100 μl of 2% formalin, and subjected to FACS analysis. FIG. 3A shows human BMSCs at P2. FACS data for CD105 (endoglin) expression is shown in FIG. 3E.

To assess the potential of human BMSCs for osteogenic and chondrogenic differentiation, the cells were cultured in pellets in either control medium (DMEM supplemented with 10% FBS, Pen-Strep and Fungizone®), chondrogenic medium (control medium supplemented with 0.1 mM nonessential amino acids, 50 μg/ml ascorbic acid-2-phosphate, 10 nm dexamethasone, 5 μg/ml insulin, 5 ng/ml TGF β1) or osteogenic medium (control medium supplemented with 50 μg/ml ascorbic acid-2-phosphate, 10 nm dexamethasone, 7 mM β-glycerophosphate, and 1 μg/ml BMP-2). Cells were isolated from monolayers by trypsin and washed in PBS. Aliquots containing 2×10⁵ cells were centrifuged at 300×g in 2 ml conical tubes and allowed to form compact cell pellets over 24 hours in an incubator (5% CO₂/37° C.). Medium was changed every 2-3 days. After 4 weeks of culture, pellets were washed twice in PBS, fixed in 10% neutral buffered formalin (24 hours at 4° C.), embedded in paraffin and sectioned (5 μm thick). Sections were stained for general evaluation (haematoxilin and eosin), the presence of glycosaminoglycan (GAG) (safranin O/fast green) (FIGS. 3B and 3C), and mineralized tissue (according to von Kossa in 5% AgNO for 1 hour, exposed to a 60 Watt bulb and counterstained with fast red; FIGS. 3F and 3G). In addition, the amounts of GAG (FIG. 3D) and calcium (FIG. 3H) were measured.

Porous, biocompatible, biodegradable scaffolds and hMSCs were used to engineer bone-like tissue in vitro. Different biocompatible scaffolds with the same porous microstructure were studied: collagen, silk, and silk with covalently bound RGD tripeptides (silk-RGD at 3.5±0.5 pM/cm²). Collagen was studied to assess the effects of fast degradation. Silk was studied to assess the effect of slow degradation. Silk-RGD was studied to assess the effects of enhanced cell attachment and slow degradation.

P2 hBMSCs were suspended in liquid Matrigel (7×10⁵ cells per scaffold in 10 μL Matrigel) on ice to prevent gelation. The cell suspension was then seeded, by capillary action, on scaffolds that had been prewetted by overnight incubation in DMEM. The seeded constructs were incubated in culture dishes at 37° C. for 15 min to allow gel hardening and then osteogenic medium (DMEM supplemented with 10% FBS, Pen-Strep and Fungizone®, 50 μg/ml ascorbic acid-2-phosphate, 10 nm dexamethasone, 7 mM beta-glycerophosphate, 1 μg/ml BMP-2) was added. Half of the medium was replaced every 2-3 days. Seeded constructs were cultured for up to 4 weeks in osteogenic medium.

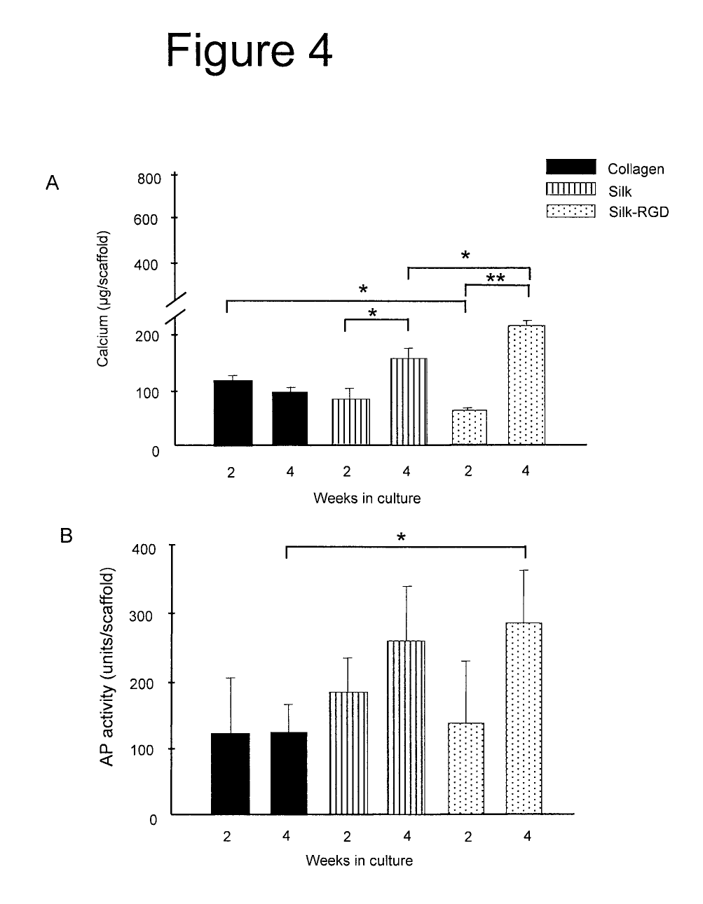

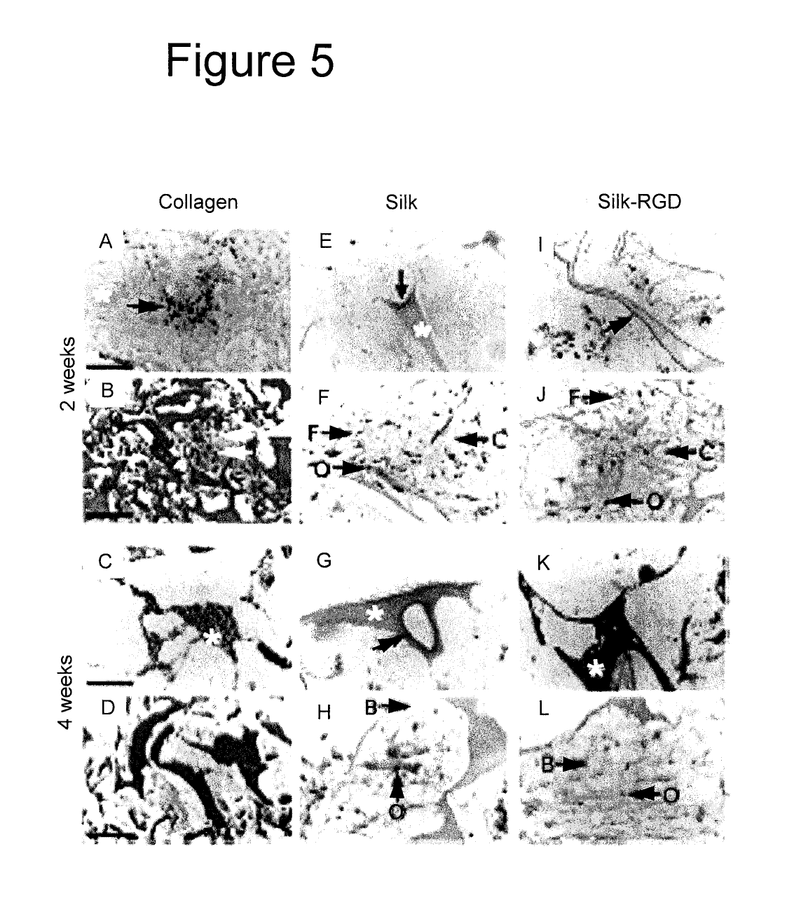

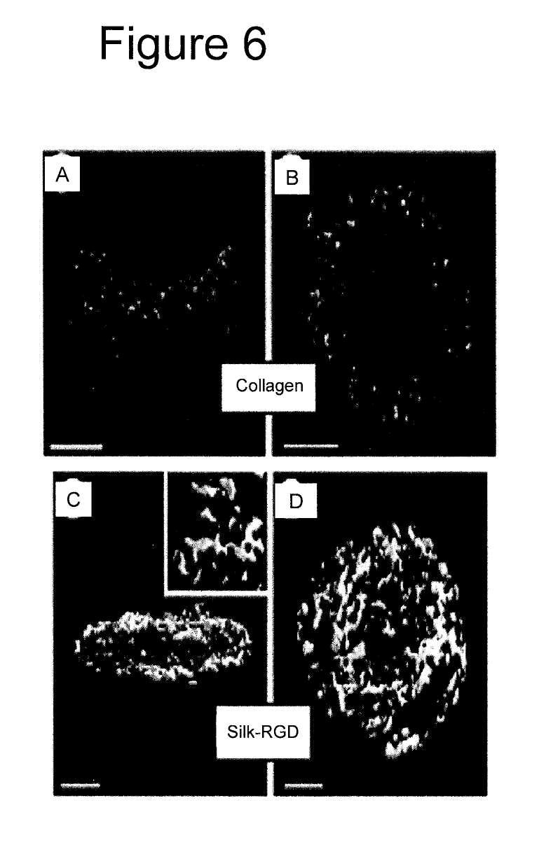

Calcium deposition (FIG. 4A) and alkaline phosphatase (AP) activity (FIG. 4B) was measured for hBMSCs on scaffolds and cultured in osteogenic medium. The data reveal increased mineralization on silk-RGD scaffolds compared to either silk or collagen scaffolds after 4 weeks. Histological analysis (FIG. 5) and MicroCT (FIG. 6) revealed the development of up to 1.2 mm long, interconnected and organized bone-like trabeculae with cuboid cells on the silk-RGD scaffolds. These features were also present on silk scaffolds, but to a lesser extent, and were absent on the collagen scaffolds. The X-ray diffraction pattern of the deposited bone corresponded to hydroxyapatite present in the native bone. Transcript expression of bone sialoprotein, osteopontin, and BMP-2 was significantly higher for hBMSC cultured in osteogenic mediums as compared to control medium, both after 2 and 4 weeks in culture.

These results support that silk-RGD scaffolds are particularly suitable for autologous and allogeneic bone tissue engineering.

### Example 5

### Human BMSCs and Silk Scaffold in Vivo

The effect of various graft/implant compositions comprising 3-dimensional silk scaffolds in accelerating healing of critical-sized calvarial bone defects was assessed in a SCID mouse model using 7 week old mice. All variants used 3-dimensional silk scaffolds except “empty defects” in which no scaffold was transplanted at all. One variant was scaffolds loaded with BMP-2 at 2.4±0.14 μg per scaffold, seeded with hBMSCs and cultured in osteogenic medium in spinner flasks for four weeks; these compositions are referred to as “tissue-engineered grafts”. A second variant was scaffolds loaded with BMP-2 at 2.4±0.14 μg per scaffold and seeded with hBMSCs but not cultured in vitro to induce differentiation; these compositions are described as “not tissue engineered”. A third variant was silk scaffolds loaded with BMP-2 but were not seeded with hBMSCs. A fourth variant was silk scaffolds that were not loaded with either BMP-2 or hBMSCs.

Scaffolds were implanted in critical size defects (4 mm in diameter). Five weeks after surgery the animals were sacrificed and bone tissues collected. The samples were processed for histology and immunohistochemical analyses using antibodies for bone markers bone sialoprotein (BSP), osteopontin (OPN) and osteocalcin (OCN). Samples were also assessed by X-ray and by MicroCT for bone mineral deposition, distribution and content.

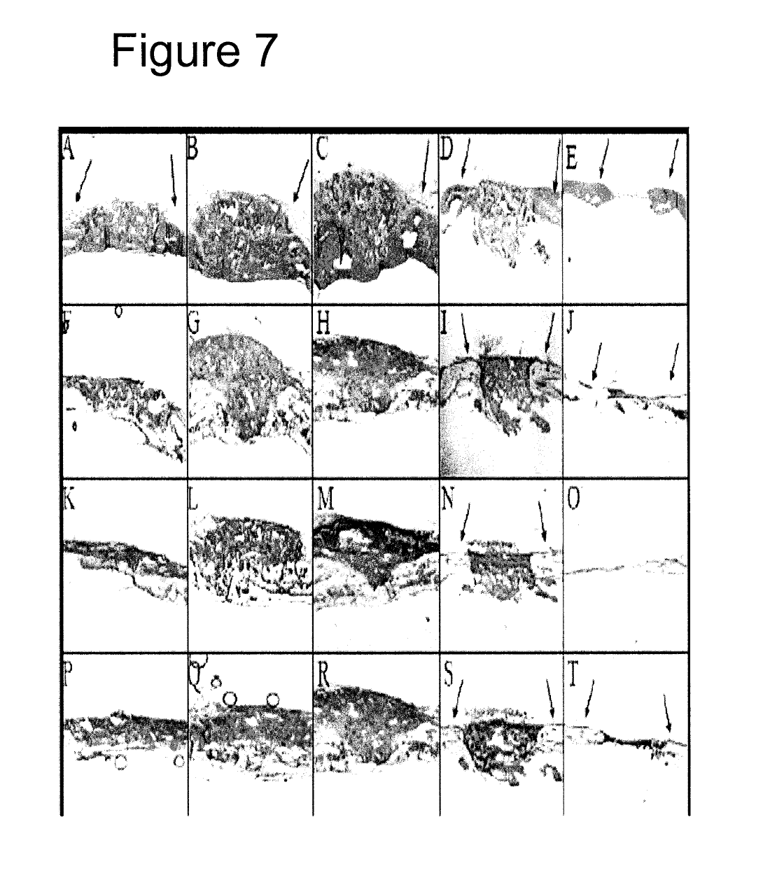

In 3-dimensional silk scaffolds loaded with BMP-2 alone (no hBMSCs), a medium amount of bone formed particularly on the endocranial side of the defect (FIGS. 7C, 7H, 7M and 7R). The rest of the wound was filled with dense cellular connective tissue which, in some cases, was protruding from the wound surface. While not being bound by theory, it is thought these cells were attracted to the wound site and might undergo differentiation into bone forming cell under the influence of BMP-2. The osteogenic potential of these cells was further demonstrated by strong immunostaining for bone marker proteins, indicating active bone formation in the wound site.

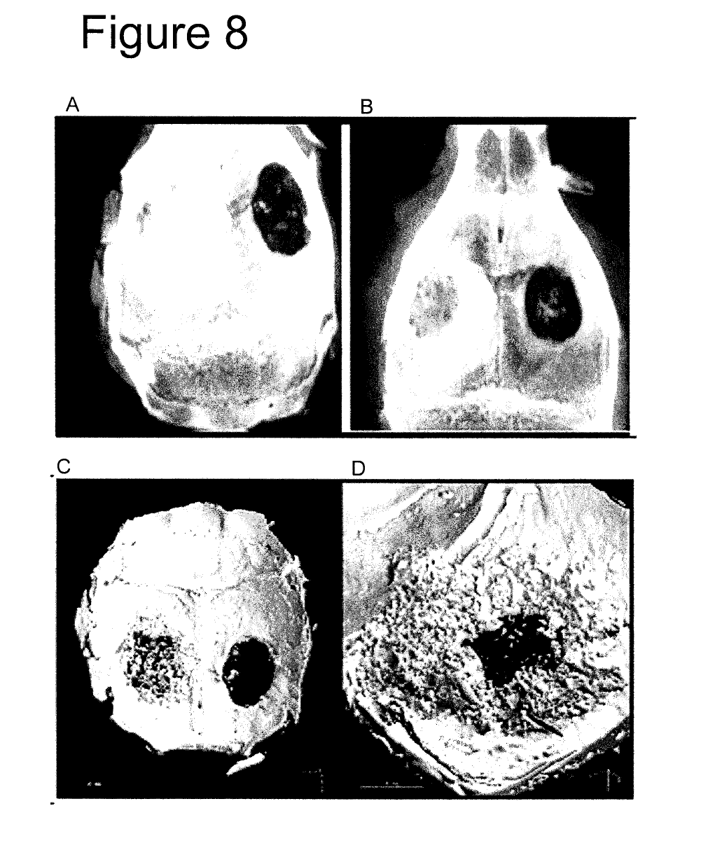

In scaffolds loaded with BMP-2 and seeded with hMSCs but not differentiated in vitro (e.g. not tissue engineered), a large amount of new bone was seen in the wound site (FIGS. 7B, 7G, 7L and 7Q). The new bone filled the wound gap. Thus the connective tissue was pushed away from the defect and could only be seen on the outer surface, capping the regenerated bone tissue. The formed bone seemed to undergo remodeling and become more organized and merge tightly to the host bone at the surgical margin. As seen in FIG. 8B, good ingrowth was detected, however, less bone was present compared to the tissue-engineered implant. In most cases, completed bony healing was seen, although residual silk scaffold remained and was seen as scattered eosin-stained strips.

Calvaria filled with tissue-engineered grafts were completely healed with newly formed bone (FIGS. 7A, 7F, 7K and 7P). Although the new bone was not as extensive as the calvaria filled with grafts of scaffolds seeded with hBMSCs but not differentiated in vitro, the new bone was, notably, more mature. Laminar bone was formed, encompassing newly formed bone marrow with hemopoietic elements. A much thinner layer of osteoid tissue was observed on the surface of new bone in this group, when compared with that in the calvaria filled with scaffolds seeded with hBMSCs but not differentiated in vitro. As shown in FIG. 8A, 8C and 8D, trabecular bone structures are visible in the graft and there was good integration of new bone from defect margins. These observations indicate the more mature nature of the bone formed in calvaria filled with tissue-engineered grafts. Immunostaining with bone-specific antibodies was positive for the calvaria filled with tissue-engineered grafts. While the mature bone, including the normal and uninjured bone, showed pale staining (except osteocytes that showed strong staining), all the cellular tissue involved in active bone formation demonstrated intense staining. The immunoreaction pattern observed was consistent with known gene expression patterns of these bone matrix proteins.

In tissues treated with the silk scaffolds alone, there was no significant bone formed (FIG. 7D, 7I, 7N and 7S). In some cases, newly-formed bone particles were observed but only in the surgical margin. The connective tissue occupying the wound gap space showed positive immunoreactions to antibodies against BSP, OPN and OCN, however, suggesting an osteoconductive effect of the silk protein. These results may also indicate that endogenous murine BMSCs present in the neighboring area migrated into and accumulated in the wound site to repair the damaged bone, and the silk scaffold provided a suitable meshwork, physically and mechanically, to accommodate these cells. While there was no mineralized solid bone seen at this stage, abundant bone matrix proteins secreted by osteoblasts were detected in the wound area.

The empty defects (sham-operated) remained open at 5 weeks postoperatively (FIGS. 7E, 7J, 7O and 7T). A thin fibrous membrane was present between the surgical incisive edges. The membrane showed some immunoreaction to the bone-specific antibodies, which might indicate there was osteogenic activity initiated by the host cells at the surgical margins. In combination with the lack of matrix support and the stimulation of growth factors, the limited number of cells at the bones' ends precluded the formation of sufficient bone to close the gap of critical sized defects.

The results from this critical size cranial defect study clearly support the value of the compositions of silk scaffolds and BMSCs to support in vivo osteogenesis and repair of bone defects. The compositions of silk scaffolds and BMSCs that were not differentiated in vitro prior to transplant surgery induced significant bone formation and defect healing. The composition of silk scaffolds and BMSCs that were differentiated in vitro prior to transplant surgery also induced significant bone forination and complete defect healing. The bone growth was more mature with these tissue-engineered grafts/implants. In addition, laminar bone, was encompassing newly formed bone marrow with hemopoietic elements, was observed with the tissue-engineered grafts.

Without being bound by theory, it is believed that the stable macroporous structure of silk-RDG scaffolds, their mechanical properties that are tailorable to better match native bone, and their slow degradation all contributed to improved bone tissue engineering using silk-RDG scaffolds.

### Example 6

### Human BMSCs and Human ASCs and Silk Scaffolds in Vitro for Adipose-Like Tissue

The ability of 3-dimensional porous HFIP-based silk scaffolds to support adipogenesis of two types of stem cell populations, hBMSCs and hASCs, was assessed. The scaffolds were made using a 17% silk fibroin (w/v). They had pore sizes of about 450 to about 550 microns and were cylinders about 5 mm in diameter and 2 mm in height. Scaffolds were seeded with either hBMSCs or ASCs (1×10⁶ cells/scaffold) and cultivated for 21 days under static culture conditions in medium consisting of DMEM supplemented with 10% FCS, 100 U/ml penicillin, 100 μg/ml streptomycin, 0.1 mM nonessential amino acids and adipogenic stimulants (AD) consisting of 0.5 mM 3-isobutyl-1-methyl-xanthine, 1 μM dexamethasone, 5 μg/ml insulin, and 50 μM indomethacin. In parallel, seeded scaffolds were cultivated identically but in medium lacking adipogenic stimulants.

Real-time RT-PCR analysis demonstrated significant upregulation of fatty acid-binding protein-4 (FABP4); lipoprotein lipase (LPL), acyl-CoA synthetase (ACS), adipsin, facilitative glucose transporter-4 (GLUT4), and peroxisome proliferator-activated receptor PPAR gamma mRNA transcript levels in both hASCs and hBMSCs in response to adipogenic stimulation, in comparison to their respective untreated controls. In addition, Oil-Red O staining of histological sections of AD-stimulated constructs revealed substantial lipid production throughout the silk scaffolds for both stem cell types. In contrast, the non-AD-induced controls did not display evidence of Oil-Red O staining.

These data indicate that compositions comprising silk scaffolds and adult stem cells (BMSCs or ASCs) support adipogenic differentiation and lipid production in vitro in response to adipogenic stimulants.

All of the above U.S. patents, U.S. patent application publications, U.S. patent applications, foreign patents, foreign patent applications and non-patent publications referred to in this specification and/or listed in the Application Data Sheet, are incorporated herein by reference, in their entirety.

While this invention has been disclosed with reference to specific embodiments, it is apparent that other embodiments and variations of this invention may be devised by others skilled in the art without departing from the true spirit and scope of the invention. The appended claims are intended to be construed to include all such embodiments and equivalent variations.

What is claimed:

1. A composition comprising a silk scaffold and an adult stem cell, wherein said adult stem cell is an adipose-derived stem cell (ASC).

2. The composition of claim 1, wherein said adult stem cell is genetically modified.

3. The composition of claim 1, wherein said adult stem cell is a human cell.

4. The composition of claim 1, wherein said ASC has been passaged up to at least a second passage.

5. The composition of claim 1, further comprising adipogenic stimulants.

6. The composition of claim 1, further comprising osteogenic stimulants.

7. The composition of claim 1, wherein said silk scaffold comprises a covalent or non-covalent modification.

8. The composition of claim 7, wherein the modification comprises at least one of RGD, parathyroid hormone (PTH) and bone morphogenetic protein-2 (BMP-2).

9. The composition of claim 1, wherein said silk scaffold has a compressive strength of about 320 KPa and a modulus of about 3330 KPa.

10. The composition of claim 1, wherein said silk scaffold is at least 90% porous and has a pore size of about 50 to about 1200 microns.

11. A method of making a composition for treating a tissue defect in a mammal, said method comprising seeding a silk scaffold with an adult stem cell to produce a seeded scaffold, wherein said adult stem cell is an adipose-derived stem cell (ASC).

12. The method of claim 11, wherein said adult stem cell is genetically modified.

13. The method of claim 11, wherein said adult stem cell is a human cell.

14. The method of claim 11, wherein said ASC has been passaged up to at least a second passage.

15. The method of claim 11, further comprising: culturing said seeded scaffold in adipogenic medium.

16. The method of claim 11, further comprising: culturing said seeded scaffold in osteogenic medium.

17. The method of claim 11, wherein said silk scaffold is made by a salt leaching process.

18. The method of claim 17, wherein said salt leaching process comprises the steps of:
extracting silk from a cocoon;
removing sericin from said extracted silk;
preparing a silk solution;
placing said silk solution in a mold containing a salt porogen;
drying said silk solution in said mold to produce a silk/porogen composite; and
extracting said porogen from said silk/porogen composite to produce a silk scaffold.

19. The method of claim 11, wherein said silk scaffold has a compressive strength of about 320 KPa and a modulus of about 3330 KPa.

20. The method of claim 11, wherein said silk scaffold is at least 90% porous and has a pore size of about 50 to about 1200 microns.

21. The method of claim 11, wherein said silk scaffold comprises a covalent or non-covalent modification.

22. The method of claim 21, wherein said modification comprises at least one of RGD, parathyroid hormone (PTH) and bone morphogenetic protein-2 (BMP-2).

23. A method of alleviating or treating a bone defect in a mammal, said method comprising administering to said mammal having a bone defect a therapeutically effective amount of a composition comprising a silk scaffold and an adult stem cell, wherein said adult stem cell is an adipose-derived stem cell (ASC) and wherein said composition is exposed to osteogenic stimulants, thereby alleviating or treating said bone defect in said mammal.

24. The method of claim 23, wherein said exposure to osteogenic stimulants occurs in osteogenic medium.

25. The method of claim 23, wherein said silk scaffold is modified.

26. The method of claim 25, wherein said modified silk scaffold comprises at least one of RGD, parathyroid hormone (PTH) and bone morphogenetic protein-2 (BMP-2).

27. The method of claim 23, wherein said silk scaffold has a compressive strength of about 320 KPa and a modulus of about 3330 KPa.

28. The method of claim 23, wherein said silk scaffold is at least 90% porous and has a pore size of about 50 to about 1200 microns.

29. The method of claim 23, wherein said ASC has been passaged up to at least a second passage.

30. The method of claim 23, wherein said ASC is a human cell.

31. The method of claim 23, wherein said mammal is a human.

32. A method of alleviating or treating a soft tissue defect in a mammal, said method comprising administering to said mammal having a soft tissue defect a composition comprising a silk scaffold and an adult stem cell, wherein said adult stem cell is an adipose-derived stem cell (ASC) and wherein said composition is been exposed to adipogenic stimulants, thereby alleviating or treating said soft tissue defect in said mammal.

33. The method of claim 32, wherein said exposure to adipogenic stimulants occurs in adipogenic medium.

34. The method of claim 32, wherein said silk scaffold is modified.

35. The method of claim 32, wherein said silk scaffold has a compressive strength of about 320 KPa and a modulus of about 3330 KPa.

36. The method of claim 32, wherein said silk scaffold is at least 90% porous and has a pore size of about 50 to about 1200 microns.

37. The method of claim 32, wherein said ASC has been passaged up to at least a second passage.

38. The method of claim 32, wherein said ASC is a human cell.

39. The method of claim 32, wherein said mammal is human.
